(19) United States
(12) Patent Application Publication
(10) Pub. No.: US 2015/0031577 A1
(43) Pub. Date: Jan. 29, 2015

(54) NUCLEIC ACID DETECTION METHOD COMPRISING TARGET SPECIFIC INDEXING PROBES (TSIP) COMPRISING A RELEASABLE SEGMENT TO BE DETECTED VIA FLUORESCENCE WHEN BOUND TO A CAPTURE PROBE

(71) Applicant: UNIVERSITÉ LAVAL, Québec (CA)

(72) Inventors: Karel Boissinot, Quebec (CA); Régis Peytavi, Cabestany (FR); Laurie Girard, Quebec (CA)

(21) Appl. No.: 14/386,588

(22) PCT Filed: Mar. 20, 2013
(86) PCT No.: PCT/CA2013/050228

§ 371 Date: Sep. 19, 2014

Related U.S. Application Data
(60) Provisional application No. 61/613,109, filed on Mar. 20, 2012.

(51) Int. Cl. C12Q 1/68

(52) U.S. Cl. CPC C12Q 1/6823; C12Q 1/6818
USPC 506/9; 435/6.11; 536/24.3

(57) ABSTRACT

Reagents, systems and methods for the detection of nucleic acids are described herein, including such methods which may be performed in a single reaction. Such reagents include a target specific indexing probe (TSIP) synthetic DNA structure comprising a detectable moiety and a quencher thereof, wherein in the presence of a target nucleic acid a portion (a tag) of the (TSIP) synthetic DNA structure comprising the detectable moiety is released, thereby increasing the signal emitted therefrom. The tag may in turn bind to a capture probe, facilitating signal detection.

### CROSS REFERENCE TO RELATED APPLICATIONS

This application claims the benefit of U.S. provisional application Ser. No. 61/613,109, filed on Mar. 20, 2012, which is incorporated herein by reference in its entirety.

### STATEMENT REGARDING FEDERALLY SPONSORED RESEARCH OR DEVELOPMENT

This invention was made with Government support under NIH Grant No. R01 A1089541-01. The Government has certain rights in this invention.

### TECHNICAL FIELD

The present invention generally relates to the detection of nucleic acids, and more specifically to the detection of a target nucleic acid which may be performed in a single reaction.

### BACKGROUND ART

Molecular diagnostic technologies using multiplex detection of nucleic acids have several applications including cancer and infectious disease diagnostics. Infectious diseases in particular require rapid response and close to patient monitoring capacity. Currently used multiplex detection methods, especially in microbiology, require a long sample-to-answer time and qualified personnel. As a result, first-line management of patients or unknown samples is mostly empirical.

Multiplex detection of target nucleic acids requires many steps. Because nucleic acids are generally encased inside cells or viral particles they must first be released and separated from cellular debris and freed of amplification inhibitors. Amplification of targets is usually performed by enzymatic replication of nucleic acids, most commonly PCR. Real-time PCR is restricted by the number of different fluorophores currently available, which limits the number of possible targets for multiplex detection. For detection of more than 5 or 6 targets, spatial confinement of targets is often used. Microarrays made of immobilized capture probes with known Cartesian coordinates (x,y) that specifically hybridize with targets can be used to increase multiplex detection.

The detection of amplicons on microarrays increases multiplex detection capacity but requires several technical steps. In most DNA microarray technologies, amplification and/or fluorescent dye labeling of the targets is required. To avoid interference with the probe, the DNA strand complementary to the labeled strand may be digested prior to hybridization. After mixing with hybridization buffer, labeled amplicons are hybridized onto the microarray. It is also necessary to wash the hybridized microarray to obtain a good signal to background ratio which increases the complexity and cost of the procedure.

There is thus a need for novel reagents, methods and systems for the detection of target nucleic acids in a sample.

The present description refers to a number of documents, the content of which is herein incorporated by reference in their entirety.

### SUMMARY OF THE INVENTION

In a first aspect, the present invention provides a method for detecting the presence of a target nucleic acid in a sample, the method comprising:
(a) contacting a sample with amplification and hybridization reagents, wherein said amplification and hybridization reagents comprises a target specific indexing probe (TSIP) synthetic DNA structure comprising a first segment, a second segment and a third segment, wherein:
   (i) the first segment comprises a sequence complementary to a first portion of the target nucleic acid and complementary to the third segment;
   (ii) the second segment is attached to the 3′ end of the first segment and comprises a sequence that is complementary to a second portion of the target nucleic acid that is contiguous to the first portion, but not complementary to the first or third segment;
   (iii) the third segment is attached to the 3′ end of the second segment and comprises
      a first part comprising a sequence that is complementary to the first segment and to a capture probe present in said reaction;
      a first part comprising a sequence that is complementary to the first segment, and a second part comprising a sequence that is complementary to the capture probe; or
      a first part comprising a sequence that is complementary to the first segment and to a first portion of the capture probe, and a second part comprising a sequence that is complementary to a second portion of the capture probe,
wherein the third segment comprises a detectable label attached thereto, and said first and/or second segment comprises a quenching moiety for the detectable label, wherein the detectable label and the quenching moiety are located within sufficiently close proximity of each other in the TSIP synthetic DNA structure such that the signal emitted by the detectable label is reduced as compared to a corresponding signal emitted by a detectable label in a corresponding TSIP synthetic DNA structure lacking said quencher moiety;
(b) performing an amplification and hybridization reaction, wherein said third segment is released from the first and second segment if the target nucleic acid is present; and
(c) detecting the hybridization of the third segment to the capture probe by virtue of an increase in the signal emitted from the detectable moiety relative to the signal emitted from a corresponding TSIP that has not been contacted with the sample, wherein the detection of an hybridization is indicative that the target nucleic acid is present in the sample.

In another aspect, the present invention provides a target nucleic acid detection kit or system comprising: a first segment, a second segment and a third segment, wherein: (i) the first segment comprises a sequence complementary to a first portion of the target nucleic acid and complementary to the third segment; (ii) the second segment is attached to the 3′ end of the first segment and comprises a sequence that is complementary to a second portion of the target nucleic acid that is contiguous to the first portion, but not complementary to the first or third segment; (iii) the third segment is attached to the 3′ end of the second segment and comprises
a first part comprising a sequence that is complementary to the first segment and to a capture probe present in said reaction;
a first part comprising a sequence that is complementary to the first segment, and a second part comprising a sequence that is complementary to the capture probe; or
a first part comprising a sequence that is complementary to the first segment and to a first portion of the capture probe, and a second part comprising a sequence that is complementary to a second portion of the capture probe,
   wherein the third segment comprises a detectable moiety attached thereto, and said first and/or second segment comprises a quenching moiety for the detectable label, wherein the detectable label and the quenching moiety are located within sufficiently close proximity of each other in the TSIP synthetic DNA structure such that the signal emitted by the detectable label is reduced as compared to a corresponding signal emitted by a detectable label in a corresponding TSIP synthetic DNA structure lacking said quencher moiety. In an embodiment, the kit further comprises a capture probe comprising a sequence complementary to at least a portion of the third segment of the TSIP synthetic DNA structure. In an embodiment, the above-mentioned kit or system further comprises a solid support, an amplification reagent, a hybridization reagent, a detection reagent, an amplification system and/or a detection system. In an embodiment, the kit or system further comprises instructions for detecting a target nucleic acid in a sample.

In an embodiment, the above-mentioned first segment comprises from about 8 to about 20 nucleotides, in a further embodiment, from about 10 to about 16 nucleotides.

In an embodiment, the above-mentioned second segment comprises from about 8 to about 40 nucleotides, in a further embodiment, from about 10 to about 30 nucleotides.

In an embodiment, the above-mentioned first and second segments comprise from about 16 to about 60 nucleotides, in a further embodiment from about 20 to about 40 nucleotides or from about 24 to about 32 nucleotides, in total.

In an embodiment, the above-mentioned third segment comprises from about 8 to about 30 nucleotides, in a further embodiment from about 14 to about 24 nucleotides.

In an embodiment, the above-mentioned capture probe comprises from about 10 to about 30 nucleotides, in a further embodiment from about 16 to about 24 nucleotides.

In an embodiment, the quenching moiety is attached to the first segment. In a further embodiment, the quenching moiety is attached to the 5′ end the first segment.

In an embodiment, the detectable label is attached to the 3′ end of the third segment.

In another embodiment, the detectable label is located at 20 nucleotides or less from the quencher. In a further embodiment, the detectable label is located at 17 nucleotides or less from the quencher.

In an embodiment, the detectable label and the quenching moiety are at a distance of about 10 nm or less.

In a further embodiment, the third segment comprises a first part comprising a sequence that is complementary to the first segment and to a first portion of the capture probe, and a second part comprising a sequence that is complementary to a second portion of the capture probe.

In a further embodiment, the second part is attached to the 3′ end of said first part, and said TSIP synthetic DNA structure is of formula I, II, III or IV:

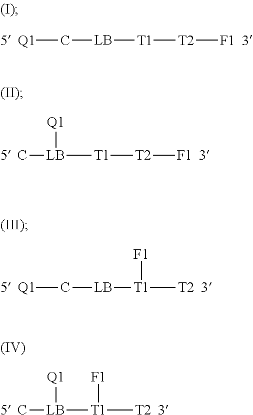

where — represent a bond, Q1 represents quenching moiety, C represents said first segment, LB represents said second segment, T1 represents the first part of the third segment, T2 represents the second part of the third segment, and F1 represents the detectable label.

In another embodiment, the second part is attached to the 5′ end of said first part, and said TSIP synthetic DNA structure is of formula V, VI, VII or VIII:

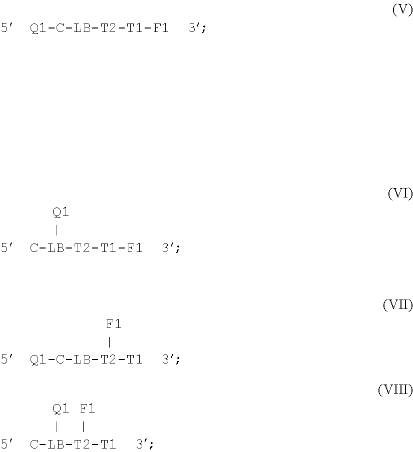

where —, Q1, C, LB, T1, T2 and F2 are as defined above.

In another embodiment, the second part is inserted within said first part, and said TSIP synthetic DNA structure is of formula IX, X, XI, XII, XIII or XIV

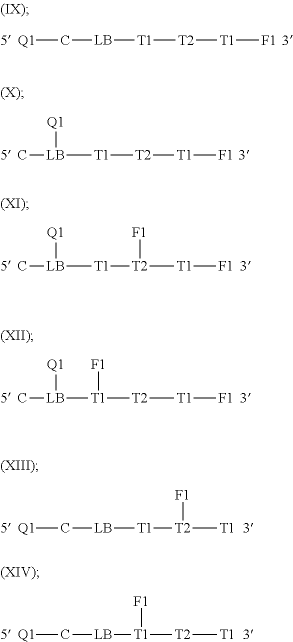

where —, Q1, C, LB, T1, T2 and F2 are as defined

In an embodiment, the detectable label is a fluorophore, in a further embodiment, indo-5-carbocyanine N-ethyl-N′-hexylamido-ethoxyethyl-O-(2-cyanoethyl)-(N,N-diisopropyl)-phosphoramidite (Quasar® 670 Amidite). In an embodiment, the quenching moiety is 4′-(4-Nitro-phenyldiazo)-2′-methoxy-5′-methoxy-azobenzene-4″-(N-2-oxy ethyl (4,4′ dimethoxy trityl))-N-ethyl-2-cyanoethyl-(N,N-diisopropyl)-phosphoramidite (Black Hole® Quencher 2).

In another embodiment, the fluorophore is tetramethylrhodamine (TAMRA). In an embodiment, the quenching moiety is Iowa Black® Red Quencher.

In an embodiment, the capture probe comprises a fluorophore suitable for fluorescence resonance energy transfer (FRET) with the fluorophore attached to the third segment.

In an embodiment, the amplification and hybridization reagents further comprise an amplification enzyme with 5′ exonuclease activity.

In another embodiment, the third segment further comprises a sequence recognized by a restriction endonuclease, and wherein the amplification and hybridization reagents further comprise the restriction endonuclease.

In an embodiment, the capture probe is attached to a solid support.

In an embodiment, the amplification and hybridization reaction occurs in a single reaction vessel.

In an embodiment, the amplification occurs in a liquid phase and the hybridization/detection occurs in a solid phase.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows the structure of an exemplary target specific indexing probe (TSIP) synthetic DNA structure;

FIG. 2 shows a schematic of the amplification probe and target competition functions of the exemplary TSIP synthetic DNA structure of FIG. 1 where in A) an amplification target is present and in B) no amplification target is present;

FIG. 3 shows the hybridization of the synthetic molecular tag segment T1[ ]-T2[ ]-F1oligonucleotide in five amplification buffers;

FIG. 4 shows real-time nucleic acid amplification resulting in TSIP synthetic DNA structure digestion;

FIG. 5 shows a hybridization comparison between amplification-digested TSIP synthetic DNA structure versus positive and negative controls; and

FIG. 6 is a schematic of the amplification probe and target competition functions of an exemplary TSIP synthetic DNA structure using fluorescence resonance energy transfer (FRET) where in A) an amplification target is present and in B) no amplification target is present;

FIG. 7 shows the structure of another exemplary TSIP synthetic DNA structure;

FIG. 8 is a schematic of the amplification probe and target competition functions of the exemplary TSIP synthetic DNA structure of FIG. 7 where in A) an amplification target is present and in B) no amplification target is present;

FIG. 9 shows the structure of another exemplary TSIP synthetic DNA structure;

FIG. 10 is a schematic of the amplification probe and target competition functions of the exemplary TSIP synthetic DNA structure of FIG. 9 where in A) an amplification target is present and in B) no amplification target is present;

FIG. 11 shows the structure of another exemplary TSIP synthetic DNA structure;

FIG. 12 is a schematic of the amplification probe and target competition functions of the exemplary TSIP synthetic DNA structure of FIG. 11 where in A) an amplification target is present and in B) no amplification target is present;

FIG. 13 shows the hybridization of the TSIP of SEQ ID NO: 1 in a single vessel for both amplification and hybridization;

FIG. 14 shows the hybridization of the TSIP of SEQ ID NOs: 13 (target1) and 17 (target2) in a single vessel for both amplification and hybridization. White bar: amplification/hybridization was carried on a positive sample for target1. Grey bar: amplification/hybridization was carried on a positive sample for target2. Horizontal lines bar: amplification/hybridization was carried in absence of both target1 and target2.

### DISCLOSURE OF INVENTION

As used in the specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. The term “about” is used to indicate that a value includes an inherent variation of error for the device or the method being employed to determine the value, or encompass values close to the recited values, for example within 10% of the recited values (or range of values). Use of the singular forms “a,” “an,” and the include plural references unless the context clearly dictates otherwise. Where a value is explicitly recited, it is to be understood that values which are about the same quantity or amount as the recited value are also within the scope of the present disclosure, as are ranges based thereon. Unless defined otherwise, all technical and scientific terms as used herein have the same meaning as commonly understood to one of ordinary skill in the art.

Disclosed herein is a nucleic acid detection method, and systems and reagents for carrying out the method, where a target specific indexing probe (TSIP) synthetic DNA structure (nanobiomachine) is capable of combining nucleic acid amplification (e.g., PCR amplification) and microarray hybridization in a single reaction and operating in a single buffer. This TSIP synthetic DNA structure is a labeled oligonucleotide designed with different target sequences and secondary structures. First, it serves as a probe during amplification, which triggers its irreversible structural modification if a specific nucleic acid target is present. The TSIP synthetic DNA structure may be partially digested during elongation resulting in the release of a molecular sequence tag designed to hybridize only to a specific probe, for example a specific probe bound onto an adjacent microarray. Second, the molecular sequence tag released during amplification hybridizes to a capture probe, while non-modified TSIP synthetic DNA structure hybridizes weakly or not at all.

Accordingly, in a first aspect, the present invention provides a method for detecting a target nucleic acid in a reaction, such as a single reaction. The method may comprise contacting a sample suspected to contain the target nucleic acid with amplification and hybridization reagents, amplifying the target nucleic acid thereby releasing a tag product, hybridizing the tag product to a complementary capture probe and/or detecting hybridized material, whereby detection of hybridized material indicates the presence of the target nucleic acid.

In another aspect, the present invention provides a method for detecting the presence of a target nucleic acid in a sample, the method comprising:
(a) contacting a sample with amplification and hybridization reagents, wherein said amplification and hybridization reagents comprise a target specific indexing probe (TSIP) synthetic DNA structure comprising a first segment, a second segment and a third segment, wherein:
   (i) the first segment comprises a sequence complementary to a first portion of the target nucleic acid and complementary to the third segment;
   (ii) the second segment is attached to the 3′ end of the first segment and comprises a sequence that is complementary to a second portion of the target nucleic acid that is contiguous to the first portion, but not complementary to the first or third segment;
   (iii) the third segment is attached to the 3′ end of the second segment and comprises:
      a first part comprising a sequence that is complementary to the first segment and to a capture probe present in said reaction;
      a first part comprising a sequence that is complementary to the first segment, and a second part comprising a sequence that is complementary to the capture probe; or
      a first part comprising a sequence that is complementary to the first segment and to a first portion of the capture probe, and a second part comprising a sequence that is complementary to a second portion of the capture probe,
   wherein the third segment comprises a detectable label attached thereto, and said first and/or second segment comprises a quenching moiety for the detectable label, wherein the detectable label and the quenching moiety are located within sufficiently close proximity of each other in the TSIP synthetic DNA structure such that the signal emitted by the detectable label is reduced as compared to a corresponding signal emitted by a detectable label in a corresponding TSIP synthetic DNA structure lacking said quencher moiety;
(b) performing an amplification and hybridization reaction, wherein said third segment is released from the first and second segment if the target nucleic acid is present; and
(c) detecting the hybridization of the third segment to the capture probe by virtue of an increase in the signal emitted from the detectable moiety relative to the signal emitted from a corresponding TSIP that has not been contacted with the sample, wherein the detection of a hybridization is indicative that the target nucleic acid is present in the sample.

TSIP Synthetic DNA Structure

The sequence and length of each segment/part of the TSIP synthetic DNA structure may be adjusted based on the particular situation/condition (e.g., melting temperature (Tm), G/C content, specificity, sensitivity, complementary primer sequence, region of interest in the target nucleic acid, etc.) and ultimately on the particular use thereof and adapted accordingly by the person of ordinary skill.

In an embodiment, the first segment comprises from about 7 or 8 to about 30 nucleotides, in embodiments from about 8 to about 25, from about 10 to about 20 nucleotides, from about 10 to about 18 nucleotides, or from about 10 to about 16 nucleotides, for example 10, 11, 12, 13, 14, 15 or 16 nucleotides.

In an embodiment, the second segment comprises from about 7 or 8 to about 50 nucleotides, in embodiments from about 8 to about 40, from about 10 to about 35 nucleotides, from about 10 to about 30 nucleotides, or from about 14 to about 30 nucleotides, for example 14, 15, 16, 17, 18, 19, 20, 21, 22, 23, 24, 25, 26, 27, 28, 29 or 30 nucleotides.

In an embodiment, the first and second segments comprise from about 14 to about 80 nucleotides, in embodiments from about 20 to about 70, from about 20 to about 60 nucleotides, from about 20 to about 50 nucleotides, from about 24 to about 40 nucleotides, or from about 24 to about 35 nucleotides, for example 24, 25, 26, 27, 28, 29, 30, 31, 32, 33, 34 or 35 nucleotides. In an further embodiment, the first and second segments comprise from about 26 to about 30 nucleotides, for example 26, 27, 28, 29 or 30. In another embodiment, the first and second segments are designed so as to have a melting temperature (Tm) of about 65° C. to about 75° C. on the target nucleic acid, in further embodiments a Tm of about 67° C. to about 73° C. or about 68° C. to about 72° C., for example 68, 69, 70, 71 or 72° C.

In an embodiment, the third segment comprises from about 7 or 8 to about 40 nucleotides, in embodiments from about 10 to about 35, from about 10 to about 30 nucleotides, from about 10 to about 25 nucleotides, or from about 16 to about 24 nucleotides, for example 16, 17, 18, 19, 20, 21, 22, 23 or 24 nucleotides.

In an embodiment, the first part of the third segment (which comprises a sequence that is complementary to the first segment) has a length that is substantially identical to the first segment. In an embodiment, the first part of the third segment comprises from about 7 or 8 to about 30 nucleotides, in further embodiments from about 8 to about 25, from about 10 to about 20 nucleotides, from about 10 to about 18 nucleotides, or from about 10 to about 16 nucleotides, for example 10, 11, 12, 13, 14, 15 or 16 nucleotides.

In an embodiment, the second part of the third segment, if present, comprises from about 2 to about 20 nucleotides, in further embodiments from about 1 to about 15 nucleotides, from about 2 to about 12 nucleotides, from about 3 to about 10 nucleotides or from about 4 to about 9 nucleotides, for example, 4, 5, 6, 7, 8 or 9 nucleotides.

In an embodiment, the TSIP synthetic DNA structure comprises from about 30 to about 120 nucleotides, in embodiments from about 30 to about 100 nucleotides, from about 35 to about 80 nucleotides, from about 35 to about 70 nucleotides, from about 40 to about 60 nucleotides or from about 45 to about 60 nucleotides, for example 45, 46, 47, 48, 49, 50, 51, 52, 53, 54, 55, 56, 57, 58, 59 or 60 nucleotides.

The first, second and third segments may be joined via any type of covalent bonds. In an embodiment the first, second and third segments are joined via phosphodiester bonds.

Examples of Configurations of the TSIP Synthetic DNA Structure

Configuration A

In an embodiment, the third segment comprises a first part comprising a sequence that is complementary to the first segment and to a capture probe present in said reaction. This embodiment may be illustrated as follows:

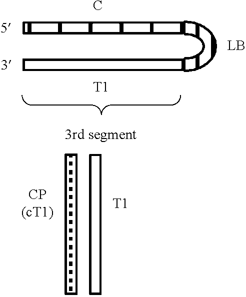

where
C represents the first segment as defined above,
LB represents said second segment as defined above,
T1 represents the third segment as defined above, and
CP represents the capture probe, which comprises a sequence complementary to T1 (cT1).

Configuration B

In another embodiment, the third segment comprises a first part comprising a sequence that is complementary to the first segment, and a second part comprising a sequence that is complementary to the capture probe. A first example (configuration B1) of this embodiment may be illustrated as follows:

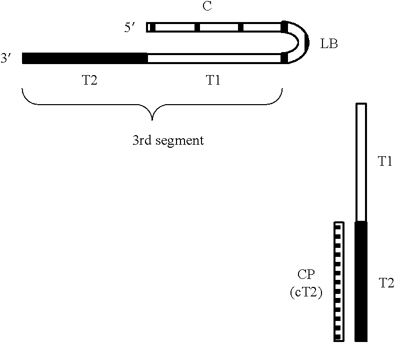

where
C represents the first segment as defined above,
LB represents said second segment as defined above,
T1 represents the first part of third segment as defined above,
T2 represents the second part of third segment as defined above, and
CP represents the capture probe, which comprises a sequence complementary to T2 (cT2).

It should be understood that according to this embodiment, the relative position of T1 and T2 in the molecule may be inverted, so that T2 is attached to the 3′ end of LB and forms part of the loop. Thus, a second example (configuration B2) of this embodiment may be illustrated as follows:

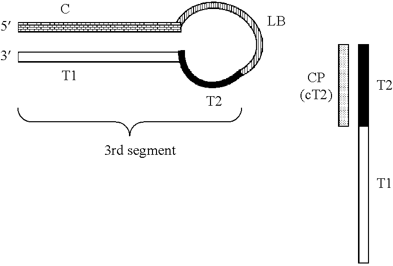

where
C represents the first segment as defined above,
LB represents said second segment as defined above,
T1 represents the first part of third segment as defined above,
T2 represents the second part of third segment as defined above, and
CP represents the capture probe, which comprises a sequence complementary to T2 (cT2).

It should further be understood that according to this embodiment, T2 may be inserted within T1 (the first part is then divided into two sections located on each side of the second part). Thus, a third example (configuration B3) of this embodiment may be illustrated as follows:

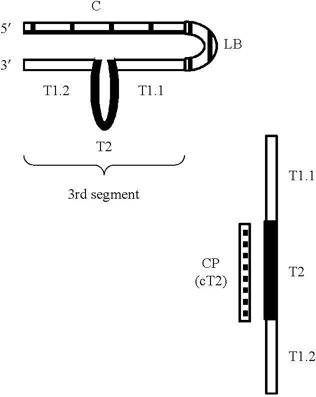

where
C represents the first segment as defined above,
LB represents said second segment as defined above,
T1.1 and T1.2 represent the first part of third segment as defined above
T2 represents the second part of third segment as defined above, and
CP represents the capture probe, which comprises a sequence complementary to T2 (cT2).

Configuration C

In another embodiment, the third segment comprises a first part comprising a sequence that is complementary to the first segment and to a first portion of the capture probe, and a second part comprising a sequence that is complementary to a second portion of the capture probe. A first example (configuration C1) of this embodiment may be illustrated as follows:

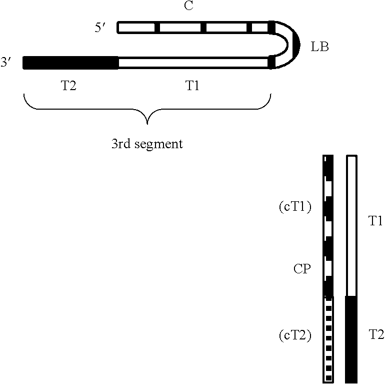

where
C represents the first segment as defined above,
LB represents said second segment as defined above,
T1 represents the first part of third segment as defined above,
T2 represents the second part of third segment as defined above, and
CP represents the capture probe, which comprises a sequence complementary to T1 (cT1) and a sequence complementary to T2 (cT2).

Again, it should be understood that according to this embodiment, the relative position of T1 and T2 in the molecule may be inverted, so that T2 is attached to the 3′ end of LB and forms part of the loop. A second example (configuration C2) of this embodiment may be illustrated as follows:

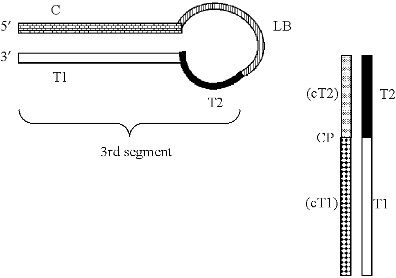

where
C represents the first segment as defined above,
LB represents said second segment as defined above,
T1 represents the first part of third segment as defined above,
T2 represents the second part of third segment as defined above, and
CP represents the capture probe, which comprises a sequence complementary to T1 (cT1) and a sequence complementary to T2 (cT2).

Also, it should further be understood that in this embodiment, T2 may be inserted within T1. Thus, a third example of this embodiment (configuration C3) may be illustrated as follows:

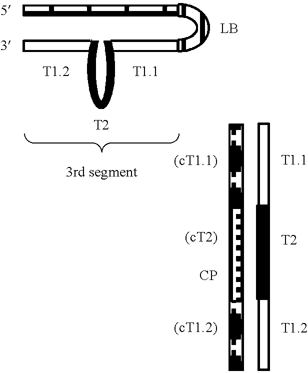

where
C represents the first segment as defined above,
LB represents said second segment as defined above,
T1.1 and T1.2 represent the first part of third segment as defined above,
T2 represents the second part of third segment as defined above, and
CP represents the capture probe, which comprises a sequence complementary to T1.1 (cT1.1), a sequence complementary to T1.2 (cT1.2) and a sequence complementary to T2 (cT2).

It should be understood that according to all the embodiments of the present invention, the capture probe may comprises sequences that are complementary to only a portion of T1, T2, T.1.1, and T1.2. An example of this option may be illustrated as follows:

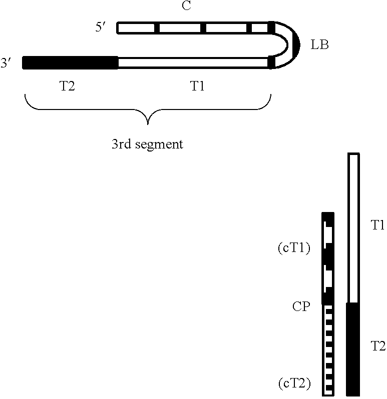

where

C represents the first segment as defined above,

LB represents said second segment as defined above,

T1 represents the first part of third segment as defined above,

T2 represents the second part of third segment as defined above, and

CP represents the capture probe, which comprises a sequence complementary to only a portion of T1 (cT1), and a sequence complementary to T2 (cT2).

Furthermore, in the configuration in which T2 is inserted within T1, the capture probe may comprise (i) a sequence that is complementary to T2 and to T1.1 (but not a sequence that is complementary to T1.2), or (ii) a sequence that is complementary to T2 and to T1.2 (but not a sequence that is complementary to T1.1). The just-noted option (ii) may be illustrated as follows:

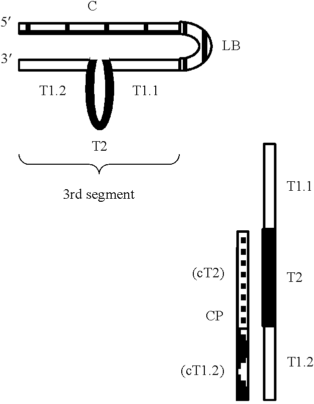

where
C represents the first segment as defined above,
LB represents said second segment as defined above,
T1.1 and T1.2 represent the first part of third segment as defined above,
T2 represents the second part of third segment as defined above, and
CP represents the capture probe, which comprises a sequence complementary to T1.2 (cT1.2) and a sequence complementary to T2 (cT2).

It should be understood that the second part (T2) may comprise two or more portions inserted within the first part T1, as follows:

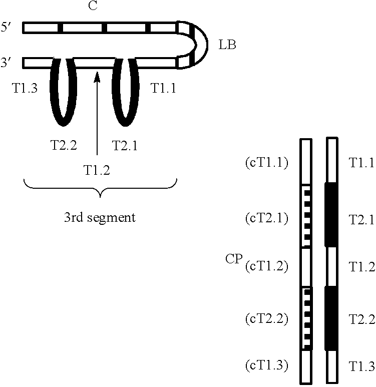

where
C represents the first segment as defined above,
LB represents said second segment as defined above,
T1.1, T1.2 and T3 represent the first part of third segment as defined above,
T2.1 and T2.2 represents the second part of third segment as defined above, and
CP represents the capture probe, which comprises a sequence complementary to T1.1 (cT1.1.), T1.2 (cT1.2), T1.3 (cT1.3), T2.1 (cT2.1) and T2.2 (cT2.2).

It should be understood that the second part of the third segment (T2), and/or the capture probe, may be designed at will to ensure specificity and optimal hybridization conditions. For example, when multiple target nucleic acids are detected, T2 may be designed to ensure that all the tags released from the TSIP synthetic DNA structures hybridize to their corresponding capture probes under similar hybridization conditions (e.g., at a similar Tm). Furthermore, when two or more target nucleic acids sequences exhibiting high sequence identity are detected in a sample, T2 (and the corresponding capture probe) may be designed to increase the specificity of the detection of the different target nucleic acids (i.e. two different tags will be released which may hybridize at two different positions on the microarray. In another embodiment, T2 may be designed to minimize the chance that it hybridizes to a nucleic acid that may be present in the sample, for example by performing a BLAST analysis.

In an embodiment, the TSIP synthetic DNA structure is of formula I, II, III or IV:

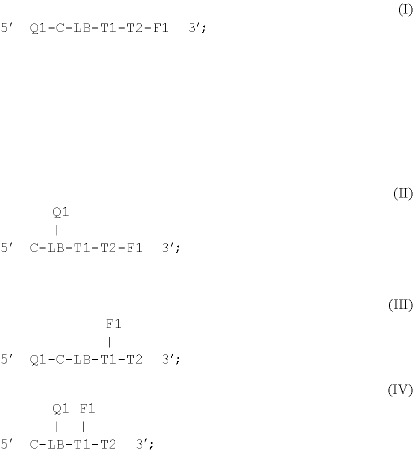

where
— represent a bond (e.g., a covalent bond),
Q1 represents quenching moiety as defined above,
C represents the first segment as defined above,
LB represents the second segment as defined above,
T1 represents the first part of the third segment as defined above,
T2 represents the second part of the third segment as defined above, and
F1 represents the detectable label as defined above,
wherein when Q1 is attached to C, it may be attached to the 5′ end or to a nucleotide within the C sequence, and when F1 is attached to T2, it may be attached to the 3′ end or to a nucleotide within the T2 sequence.

In another embodiment, the TSIP synthetic DNA structure is of formula V, VI, VII or VIII:

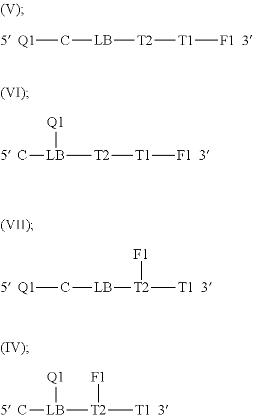

where —, Q1, C, LB, T1, T2 and F2 are as defined above, wherein when Q1 is attached to C, it may be attached to the 5′ end or to a nucleotide within the C sequence, and when F1 is attached to T1, it may be attached to the 3′ end or to a nucleotide within the T1 sequence.

In another embodiment, the TSIP synthetic DNA structure is of formula IX, X, XI, XII, XIII or XIV:

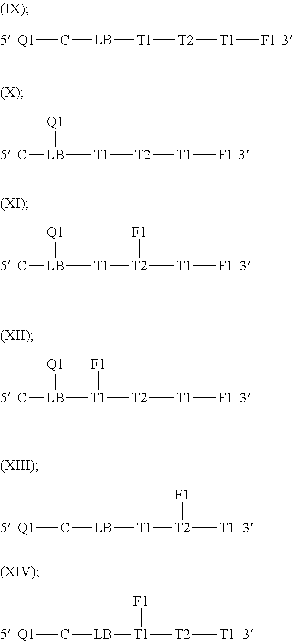

where —, Q1, C, LB, T1, T2 and F2 are as defined above, wherein when Q1 is attached to C, it may be attached to the 5′ end or to a nucleotide within the C sequence, and when F1 is attached to T1, it may be attached to the 3′ end or to a nucleotide within the T1 sequence.

In another embodiment, the TSIP synthetic DNA structure is of formula XV

Q1-C[ ]-LB[ ]-T1[ ]-T2[ ]-F1 (XV)

where
— represent bonds (e.g., phosphodiesters bonds and/or other covalent bonds),
[ ] are used to indicate the number of nucleotides for each segment,
Q1 is a quencher,
C segment is a reverse-complement to a target nucleic acid,
LB segment is complementary to a target nucleic acid but not to other parts of the TSIP synthetic DNA structure,
T1 segment is complementary to C segment and part of a capture probe,
T2 segment is complementary to the other part of the capture probe, and
F1 is a fluorophore.

In another embodiment, the TSIP synthetic DNA structure is of formula XVI

Q1-C[ ]-LB[ ]-T2[ ]-T1[ ]-F1 (XVI);

where
— represent bonds (e.g., phosphodiesters bonds and/or other covalent bonds),
[ ] are used to indicate the number of nucleotides for each segment,
Q1 is a quencher,
C segment is a reverse-complement to a target nucleic acid,
LB segment is complementary to a target nucleic acid but not to other parts of the TSIP synthetic DNA structure,
T1 segment is complementary to C segment and part of a capture probe,
T2 segment is complementary to the other part of the capture probe and it adopts a loop conformation in the TSIP synthetic DNA structure, and
F1 is a fluorophore.

In another embodiment, the TSIP synthetic DNA structure is of formula XVII

Q1-C[ ]-LB[ ]-T1.1[ ]-T2[ ]-T1.2[ ]-F1 (XVII);

where
— represent bonds (e.g., phosphodiesters bonds and/or other covalent bonds),
[ ] are used to indicate the number of nucleotides for each segment,
Q1 is a quencher,
C segment is a reverse-complement to a target nucleic acid,
LB segment may be complementary to a target nucleic acid but not to other parts of the TSIP synthetic DNA structure,
T1.1 segment is complementary to part of the C segment,
T1.2 segment is complementary to part of the C segment,
T2 segment is complementary to the microarray capture probe and it adopts a loop conformation in the TSIP synthetic DNA structure, and
F1 is a fluorophore.

In another embodiment, the TSIP synthetic DNA structure is of formula XVIII

C([ ]-Q1-[ ])-LB[ ]-T1.1([ ]-F1-[ ])-T2[ ]-T1.2[ ] (XVIII);

where
— represent bonds (e.g., phosphodiesters bonds and/or other covalent bonds),
[ ] are used to indicate the number of nucleotides for each segment,
( ) are used to indicate 2 parts of the same segment, where a modification is inserted,
C segment is a reverse-complement to a target nucleic acid and contains Q1,
Q1 is a quencher, in a lateral position of a thymine nucleotide,
LB segment may be complementary to a target nucleic acid but not to other parts of TSIP synthetic DNA structure,
T1.1 segment is complementary to part of the C segment and contains F1 (and may be complementary to a part of a capture probe),
F1 is a fluorophore, attached to a thymine nucleotide,
T1.2 segment is complementary to part of the C segment (and may be complementary to a part of a capture probe), and
T2 segment is complementary to at least a part of the capture probe (and may be dependent or independent to the target) and it adopts a loop conformation in the TSIP synthetic DNA structure.

In another embodiment, the TSIP synthetic DNA structure is of formula XIV

C1.1[ ]-t(-Q1)-C1.2[ ]-LB[ ]-T1.1.1[ ]-t(-F1)-T1.1.2[ ]-T2[ ]-T1.2[ ] (XIV);

where
— represent bonds (e.g., phosphodiesters bonds and/or other covalent bonds),
[ ] are used to indicate the number of nucleotides for each segment,
( ) are used to indicate 2 parts of the same segment, where a modification is inserted,
C1.1 segment is a reverse-complement to a target nucleic acid,
t is a thymine
Q1 is a quencher
C1.2 segment is a reverse-complement to a target nucleic acid,
LB segment may be complementary to a target nucleic acid but not to other parts of TSIP synthetic DNA structure
T1.1.1 segment is complementary to part of the C segment (and may be complementary to a part of a capture probe)
F1 is a fluorophore
T1.1.2 segment is complementary to part of the C segment (and may be complementary to a part of a capture probe)
T1.2 segment is complementary to part of the C segment (and may be complementary to a part of a capture probe), and
T2 segment is complementary to the microarray capture probe (and may be dependent or independent to the target) and it adopts a loop conformation in the TSIP synthetic DNA structure.

The terms “complementary” or “complementarity” refer to the natural binding of polynucleotides under permissive salt and temperature conditions by base-pairing. For example, the sequence “A-G-T” binds to the complementary sequence “T-C-A”. Complementarity between two single-stranded molecules may be “partial”, or it may be complete when total complementarity exists between single-stranded molecules. The degree of complementarity between nucleic acid strands has significant effects on the efficiency and strength of hybridization between nucleic acid strands. By “sufficiently complementary” is meant a contiguous nucleic acid base sequence that is capable of hybridizing to another sequence by hydrogen bonding between a series of complementary bases. Complementary base sequences may be complementary at each position in sequence by using standard base pairing (e.g., G:C, A:T or A:U pairing) or may contain one or more residues (including abasic residues) that are not complementary by using standard base pairing, but which allow the entire sequence to specifically hybridize with another base sequence in appropriate hybridization conditions. Contiguous bases of an oligomer are preferably at least about 80% (81, 82, 83, 84, 85, 86, 87, 88, 89, 90, 91, 92, 93, 94, 95, 96, 97, 98, 99, 100%), more preferably at least about 90% complementary to the sequence to which the oligomer specifically hybridizes. “Complementary” and “Complementarity” as used herein encompass both 100% (or perfect) complementary/complementarity and partial complementary/complementarity, as long as the two sequences have sufficient complementarity to hybridize to each other at a given temperature and under conditions (e.g., under conditions typically used for nucleic acid amplification and/or hybridization).

“Hybridization” or “nucleic acid hybridization” refers generally to the hybridization of two single stranded nucleic acid molecules having complementary base sequences, which under appropriate conditions will form a thermodynamically favored double stranded structure. The term “hybridizes” as used herein may relate to hybridizations under stringent or non-stringent conditions. The setting of conditions is well within the skill of the artisan and can be determined according to protocols described in the art. The term “hybridizing sequences” preferably refers to sequences which display a sequence identity of at least 40%, preferably at least 50%, more preferably at least 60%, even more preferably at least 70%, particularly preferred at least 80%, more particularly preferred at least 90%, even more particularly preferred at least 95% and most preferably at least 97%, 98% or 99% identity. Examples of hybridization conditions can be found in laboratory manuals (Sambrook et al., 2000, and Ausubel et al., 1994), or further in Hames and Higgins (Eds.) “Nucleic acid hybridization, a practical approach” IRL Press Oxford, Washington D.C., (1985)) and are commonly known in the art. In the case of a hybridization to a nitrocellulose filter (or other such support like nylon), as for example in the well-known Southern blotting procedure, a nitrocellulose filter can be incubated overnight at a temperature representative of the desired stringency condition (60-65° C. for high stringency, 50-60° C. for moderate stringency and 40-45° C. for low stringency conditions) with a labeled probe in a solution containing high salt (6×SSC or 5×SSPE), 5×Denhardt's solution, 0.5% SDS, and 100 μg/ml denatured carrier DNA (e.g., salmon sperm DNA). It has also been determined that the nature of salts present can influence hybridization of DNA (Owczarzy et al., Biochemistry, 47:5336-5353 (2008) and \check{S}pringer et al., Nucleic Acids Research, 38:7343-7351 (2010). These studies show that divalent magnesium is more efficient in stabilizing duplexes than monovalent sodium. The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The salt and SDS concentration of the washing solutions may also be adjusted to accommodate for the desired stringency. The selected temperature and salt concentration is based on the melting temperature (Tm) of the DNA hybrid. Other protocols or commercially available hybridization kits (e.g., ExpressHyb® from BD Biosciences Clontech®) using different annealing and washing solutions can also be used as well known in the art. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. A hybridization complex may be formed in or between one nucleic acid sequence present in solution and another nucleic acid sequence immobilized on a solid support (e.g., membranes, filters, chips, beads, pins or glass slides to which, e.g., the capture probe has been fixed).

“Amplification” or “amplification reaction” refers to any in vitro procedure for obtaining multiple copies (“amplicons”) of a target nucleic acid sequence or its complement, or fragments thereof. In vitro amplification refers to production of an amplified nucleic acid that may contain less than the complete target region sequence or its complement. In vitro amplification methods include, e.g., transcription-mediated amplification, replicase-mediated amplification, polymerase chain reaction (PCR) amplification, ligase chain reaction (LCR) amplification and strand-displacement amplification (SDA including multiple strand-displacement amplification method (MSDA)).

Any reagents typically used in amplification and/or hybridization reactions may be used in the above-mentioned method. In an exemplary embodiment, amplification and hybridization reagents may comprise, without limitation, one or more primers complementary to the target nucleic acid(s), dNTPs, salts (e.g., MgCl₂), buffers, BSA, enzymes such as polymerases (examples described below), and other reagents. Also, amplification buffers/solutions (“PCR mix”) are commercially available, e.g., GoTaq® (Promega®), PC2® (KlenTaq®), AptaTaq® (Roche® Applied Sciences), Takara® Taq premix (Takara Bio®) and One-step® RT-PCR reaction mix (Invitrogen®).

Detectable Label

As used herein, the term “detectable label” refers to a moiety emitting a signal (e.g., light) that may be detected using an appropriate detection system, and which may be quenched by a suitable moiety when present in proximity thereof. The detectable label can be joined, directly or indirectly, to the TSIP synthetic DNA structure. Direct labeling can occur through bonds or interactions that link the label to the nucleic acid (e.g., covalent bonds or non-covalent interactions), whereas indirect labeling can occur through the use of a “linker” or bridging moiety, which is either directly or indirectly labeled.

Any suitable detectable label may be used in the method described herein. Detectable labels include, for example, enzyme or enzyme substrates, reactive groups, chromophores such as dyes or colored particles, luminescent moieties including a bioluminescent, phosphorescent or chemiluminescent moieties, and fluorescent moieties. In an embodiment, the detectable label is a fluorescent moiety.

As used herein, the terms “fluorophore,” “fluorescent moiety,” “fluorescent label” and “fluorescent molecule” are interchangeable and refer to a molecule, label or moiety that has the ability to absorb energy from light, transfer this energy internally, and emit this energy as light of a characteristic wavelength.

Any fluorescent label or fluorophore may be used without limitation with the methods and compositions provided herein. In some embodiments, the fluorophore may be quenched by a known quencher. In some embodiments, the fluorophore may be easily incorporated internally to the third segment of the TSIP synthetic DNA structure or may be incorporated at the 3′ end of the third segment of the TSIP synthetic DNA structure. In some embodiments, the fluorophore is a commonly used fluorophore. Fluorophores that are commonly used include, but are not limited to, fluorescein, 5-carboxyfluorescein (FAM), 2′7′-dimethoxy-4′5′-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′-dimethylaminophenylazo)benzoic acid (DABCYL), and 5-(2′-aminoethyl)aminonaphthalene-1-sulfonic acid (EDANS). The fluorophore may be any fluorophore known in the art, including, but not limited to: FAM, TET, HEX, Cy3, TMR, ROX, Texas Red®, LC red 640, Cy5, and LC red 705. Fluorophores for use in the methods and compositions provided herein may be obtained commercially, for example, from Biosearch Technologies (Novato, Calif.), Life Technologies (Carlsbad, Calif.), GE Healthcare (Piscataway N.J.), Integrated DNA Technologies (Coralville, Iowa) and Roche Applied Science (Indianapolis, Ind.). In some embodiments, the fluorophore is chosen to be usable with a specific detector, such as a specific spectrophotometric thermal cycler, depending on the light source of the instrument. In some embodiments, the fluorophore is chosen to work well with a specific quencher. In some embodiments, if the assay is designed for the detection of two or more target nucleic acids (multiplex assays), two or more different fluorophores may be chosen with absorption and emission wavelengths that are well separated from each other (i.e., have minimal spectral overlap).

The TSIP synthetic DNA structure may comprise one or a plurality (e.g., 2, 3, 4 or more) of detectable labels. In an embodiment, the TSIP synthetic DNA structure comprises a single detectable label.

Quenchers

As used herein, the terms “quencher,” “quencher moiety,” and “quencher molecule” are interchangeable and refer to a molecule, moiety, or label that is capable of quenching the signal (e.g., light) emitted by the detectable label, such as a luminescent (e.g., fluorophore) emission.

In the case of a fluorophore emission, the quenching may occur as a result of the formation of a non-fluorescent complex between the fluorophore and the quencher (i.e. the quencher does not emit any fluorescent signal). Alternatively, the quenching may occur as a result of the absorption of light emitted by the fluorophore, and emission (by the quencher) of light having a wavelength that is different than the light emitted by the fluorophore. The quenching of a fluorescent signal is commonly referred to as Fluorescence (or Förster) Resonance Energy Transfer (FRET). In FRET, energy is passed non-radioactively between a donor molecule, typically a fluorophore, and an acceptor molecule, the quencher (which may or may not be a fluorophore). The donor absorbs a photon and transfers this energy non-radioactively to the acceptor. The fluorescence of the donor molecule (first fluorophore) is quenched, while the fluorescence intensity of the acceptor molecule (a fluorophore quencher) is enhanced. When the excited-state energy of the donor is transferred to a non-fluorophore quencher, the fluorescence of the donor is quenched without subsequent emission of fluorescence by the acceptor.

Any quencher may be used without limitation in the methods and compositions provided herein. The quencher may be attached to the first or second segment of the TSIP synthetic DNA structure. In some embodiments, the quencher may be easily incorporated internally to the first or second segment of the TSIP synthetic DNA structure or may be incorporated at the 5′ end of the first segment of the TSIP synthetic DNA structure. Any quencher may be used as long as it decreases the intensity of the signal emitted by the detectable label that is being used, for example the fluorescence intensity when the detectable label is a fluorophore. Quenchers commonly used for FRET include, but are not limited to, Deep Dark® Quencher DDQ-I, DABCYL, Eclipse® Dark quencher, Iowa Black® FQ, BHQ-1, QSY-7, BHQ-2, DDQ-II, Iowa Black® RQ, QSY-21, and Black Hole Quencher® BHQ-3. Quenchers for use in the methods and compositions provided herein may be obtained commercially, for example, from Eurogentec (Belgium), Epoch Biosciences (Bothell, Wash.), Biosearch Technologies (Novato Calif.), Integrated DNA Technologies (Coralville, Iowa) and Life Technologies (Carlsbad, Calif.).

The TSIP synthetic DNA structure may comprise one or a plurality (e.g., 2, 3, 4 or more) of quenchers. In an embodiment, the TSIP synthetic DNA structure comprises a single quencher.

Fluorophore-Quencher Pairs

Pairs of fluorophore-quencher molecules that can engage in fluorescence resonance energy transfer (FRET) are termed “quencher-fluorophore pair” or “FRET pairs”. As used herein, the terms “quencher-fluorophore pair” or “FRET pair” are interchangeable and refer to a pair of FRET labels including a fluorophore and a quencher that is capable of quenching the fluorophore. Suitable FRET pairs are well known in the art, and the skilled person would be able to easily select suitable FRET pairs. Examples of specific fluorophore-quencher pairs that may be employed in the present invention include, but are not limited to, fluorescein/DABCYL, EDANS/DABCYL, CAL Fluor® Gold 540/BHQ®-I, Cy3/BHQ-1, FAM/BHQ®-1, TET/BHQ®-1, JOE/BHQ®-I, HEX/BHQ®-1, Oregon Green®/BHQ-1, Cy3/BHQ®-2, Cy5/BHQ-2, ROX/BHQ®-2, TAMRA/BHQ-2, Cy5/BHQ®-3, and Cy5.5/BHQ®-3.

In order for energy transfer to occur, the fluorophore and the quencher molecules must typically be in close proximity. The quencher and fluorophore are separated at a distance such that when there is no target nucleic acid in the sample, the fluorophore is quenched by the quencher in the TSIP synthetic DNA structure. However, when the target nucleic acid is present in the sample, the TSIP synthetic DNA structure will hybridize (via the first and second segments) to the target nucleic acid and the third segment may be released from the first and second segments (via the strategies described below), thus increasing the distance between the quencher and the fluorophore, and reducing/abolishing the quenching. The unquenched fluorescent released third segment may then hybridize to the capture probe, and the fluorescent signal may be detected to assess the presence of the target nucleic acid.

The TSIP synthetic DNA structure may comprise one or a plurality (e.g., 2, 3, 4 or more) of fluorophore-quencher pairs. In an embodiment, the TSIP synthetic DNA structure comprises a single fluorophore-quencher pair.

In an embodiment, the fluorophore and the quencher are located within close proximity of each other in the TSIP synthetic DNA structure such that fluorescence emission of the fluorophore in the TSIP synthetic DNA structure is reduced as compared to a corresponding fluorescence emission in a corresponding TSIP synthetic DNA structure lacking said quencher molecule. In an embodiment, at least 50% of the fluorescence emitted by the fluorophore is quenched. In embodiments, at least 55%, 60%, 65%, 70%, 75%, 75%, 80%, 85% or 90% of the fluorescence emitted by the fluorophore is quenched.

In an embodiment, the detectable label (e.g., fluorophore) and the quencher are at a distance of about 15 nanometers (nm) or less, for example 14, 13, 12, 11 nm or less. In a further embodiment, the detectable label (e.g., fluorophore) and the quencher are at a distance of about 10 nm or less, for example 9, 8, 7, 6 or 5 nm or less.

In some embodiments, the detectable label (e.g., fluorophore) is incorporated internally to the third segment of the TSIP synthetic DNA structure, and the quencher is incorporated internally to the first or second segment of the TSIP synthetic DNA structure.

In some embodiments, the detectable label (e.g., fluorophore) is located at 20 (linear distance in the TSIP synthetic DNA strand) nucleotides or less from the quencher (for example at 19, 18, 17, 16, 15, 14, 13, 12, 11, 10, 9, 8, 7, 6, or 5 nucleotides or less from the quencher. In an embodiment, the distance between the detectable label (e.g., fluorophore) and the quencher is between about 4 bases and 20 bases, for example between about 5 to about 17 nucleotides. Such a configuration advantageously permits to maintain the detectable label (e.g., fluorophore) and the quencher in close proximity (e.g., within about 10 nm or less) independently of the structure adopted by the TSIP synthetic DNA structure.

The detectable moiety and quencher moiety may be attached to nucleotides of TSIP synthetic DNA structure via any type of bonds (covalent or non-covalent). The detectable label (e.g., fluorophore) and quencher may thus be directly or indirectly (e.g., via a ligand molecule or a linker) attached to the TSIP synthetic DNA structure using any method known to those of skill in the art. Also, custom-made nucleic acids (probes) with covalently attached detectable label (e.g., fluorophore) and quenchers may be obtained commercially from several providers (e.g., Biosearch Technologies®, Inc. and Integrated DNA Technologies®, Inc.). In an embodiment, the quencher is attached to the 5′ end of the TSIP synthetic DNA structure (5′ end of the first segment). The 5′-end detectable label can be covalently attached at any available moiety of the 5′-end nucleotide of the first segment (the triphosphate, the nitrogenous base, or the sugar of the 5′-end nucleotide). In particular examples, the 5′-end label is covalently attached at the triphosphate of the 5′-end nucleotide. In other particular examples, the 5′-end label is covalently attached at any available moiety of the nitrogenous base of the 5′-end nucleotide. In other particular examples, the 5′-end label is covalently attached to any available moiety of the sugar component of the 5′-end nucleotide.

For example, using commercially available phosphoramidite reagents, one can produce oligonucleotides containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite, and can label them using protocols described in, for example, PCR Protocols: A Guide to Methods and Applications, ed. by Innis et al., Academic Press, Inc., 1990. Methods for incorporating oligonucleotide functionalizing reagents having one or more sulfhydryl, amino or hydroxyl moieties into the oligonucleotide reporter sequence, typically at the 5′ terminus, are described in U.S. Pat. No. 4,914,210

In an embodiment, the quencher is attached to the 3′ end of the TSIP synthetic DNA structure (3′ end of the third segment). The 3′-end quencher can be covalently attached at any available moiety of the 3′-end nucleotide of the third segment (the triphosphate, the nitrogenous base, or the sugar of the 3′-end nucleotide). In particular examples, the 3′-end quencher is covalently attached at the triphosphate of the 3′-end nucleotide. In other particular examples, the 3′-end quencher is covalently attached at any available moiety of the nitrogenous base of the 3′-end nucleotide. In other particular examples, the 3′-end quencher is covalently attached to any available moiety of the sugar component of the 3′-end nucleotide.

In an embodiment, the quencher and/or fluorophore is/are attached internally in TSIP synthetic DNA structure (e.g., to any suitable nucleotide within the TSIP synthetic DNA structure, such as a thymine). Appropriate linking methodologies for attachment of dyes to oligonucleotides are described in many references, e.g., Marshall, Histochemical J., 7: 299-303 (1975); Menchen et al., U.S. Pat. No. 5,188,934; Menchen et al., European Patent Application 87310256.0; and Bergot et al., International Application PCT/US90/05565.

Covalent attachment of the 5′-end/internal detectable label and 3′-end/internal quencher to the triphosphate, nitrogenous base, and/or sugar of a nucleotide of the TSIP synthetic DNA structure can be accomplished according to standard methodology well known in the art as discussed, for example in Sambrook et al. (Molecular Cloning: A Laboratory Manual, Cold Spring Harbor, N.Y., 2001), Ausubel et al. (In Current Protocols in Molecular Biology, John Wiley & Sons, New York, 1998), Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19:3019 (1991) (3′ sulfhydryl); Giusti et al., PCR Methods and Applications, 2:223-227 (1993) and Fung et al, U.S. Pat. No. 4,757,141 (5′ phosphoamino group via Aminolink™ II available from Applied Biosystems®, Foster City, Calif.); Stabinsky, U.S. Pat. No. 4,739,044 (3′ aminoalkylphosphoryl group); Agrawal et al., Tetrahedron Letters, 31:1543-1546 (1990) (attachment via phosphoramidate linkages); Sproat et al., Nucleic Acids Research, 15:4837 (1987) (5′ mercapto group); Nelson et al., Nucleic Acids Research, 17:7187-7194 (1989) (3′ amino group); and the like.

Modifications

The TSIP synthetic DNA structure described herein may be DNA or derivatives or modified versions thereof, so long as it is still capable of hybridizing to a target nucleic. The TSIP synthetic DNA structure may be modified at the base moiety, sugar moiety, or phosphate backbone, and may include other appending groups or labels, etc. For example, the TSIP synthetic DNA structure may comprise at least one modified base moiety such as 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and/or 2,6-diaminopurine. The TSIP synthetic DNA structure may comprise at least one modified sugar moiety such as arabinose, 2-fluoroarabinose, xylulose, and/or hexose. The TSIP synthetic DNA structure may also comprise at least one modified phosphate backbone such as a phosphorothioate, a phosphorodithioate, a phosphoramidothioate, a phosphoramidate, a phosphordiamidate, a methylphosphonate, an alkyl phosphotriester, and/or a formacetal or analog thereof. The TSIP synthetic DNA structure may be modified to more strongly bind to the target nucleic acid, the capture probe, or both. Examples of modifications that may enhance the binding of a DNA molecule include, but are not limited to, 2′-O-alkyl modified ribonucleotides, 2′-O-methyl ribonucleotides, 2′-orthoester modifications (including but not limited to 2′-bis(hydroxyl ethyl), and 2′ halogen modifications and locked nucleic acids (LNAs).

Tartlet Nucleic Acid

As used herein, a target nucleic acid refers to a nucleic acid sequence on a double or single stranded nucleic acid. A target nucleic acid may be sufficiently capable of hybridizing with the TSIP synthetic DNA structure described herein and include, without limitation, genomic DNA, cDNA, DNA digests, chromosomal DNA, plasmids, vectors and/or RNA such as mRNA and rRNA (that may be converted to DNA). In an exemplary embodiment of the present disclosure, a target nucleic acid is a pathogenic and/or non-pathogenic microbial nucleic acid including, without limitation, bacterial nucleic acid, yeast nucleic acid and/or viral nucleic acid.

Release of the Third Segment

Any strategies that permit to selectively induce the release of the third segment (the tag) from the first and second segment when the target nucleic acid is present (i.e. when the TSIP synthetic DNA structure forms a duplex with a target nucleic acid present in the sample) may be used in accordance with the instant methods. The released tag (whose detectable label is no longer quenched) may then hybridize to the capture probe, and the fluorescent signal may be detected to assess the presence of the target nucleic acid. As such, any configuration of the TSIP synthetic DNA structure which allows for its cleavage between the detectable label and the quenching moiety, such that the tag (comprising the detectable moiety) is released in the presence of the target nucleic acid, may be utilized.

For example, in a first embodiment, the second segment (at its 3′ end) may be designed to comprise a sequence recognized by a restriction endonuclease (at its 3′ end). Thus, addition of the restriction endonuclease to the reaction may cleave the TSIP synthetic DNA structure hybridized to its target nucleic acid (as the second segment now forms a duplex that may be recognized and cleaved by the restriction endonuclease), hence separating the detectable label from the quencher and permitting the hybridization of the third segment (tag) with the capture probe. In the absence of the target nucleic acid, the second segment remains single stranded in the TSIP synthetic DNA structure, and thus there is no cleavage by the restriction endonuclease.

In another embodiment, the strategy may involve the use of an enzyme with 5′ exonuclease activity for amplification. As used herein, “5′ exonuclease activity”, “5′ to 3′ exonuclease activity” or “5′→3′ exonuclease activity” refers to that activity of a template-specific nucleic acid polymerase e.g. a 5′→3′ exonuclease activity traditionally associated with some DNA polymerases whereby mononucleotides or oligonucleotides are removed from the 5′ end of a polynucleotide in a sequential manner, (i.e., E. coli DNA polymerase I has this activity whereas the Klenow fragment does not, or polynucleotides are removed from the 5′ end by an endonucleolytic activity that may be inherently present in a 5′ to 3′ exonuclease activity.

If the target sequence is present in a sample, the TSIP synthetic DNA structure will hybridize specifically to the target sequence; in this hybridized conformation, it will be accessible for digestion by a double strand-specific 5′ exonuclease during extension, thus releasing the quencher that was attached to the first or second segments (and also releasing the third segment that may then hybridize to the capture probe). The release of the quencher and the third segment from the initial TSIP synthetic DNA structure, by increasing the distance between the quencher and the fluorophore, reduces/abolishes the quenching. In the absence of the target nucleic acid, the first and second segments of TSIP synthetic DNA structure cannot hybridize to the target sequence, and digestion by a double strand specific 5′ exonuclease cannot occur. Any amplification enzyme with extension and 5′ exonuclease activity (e.g., polymerases) may be used in the methods described herein. As used herein “polymerase” refers to any enzyme having a nucleotide polymerizing activity. Polymerases useful in accordance with the present teachings include, but are not limited to, commercially available or natural DNA-directed DNA polymerases, Polymerases used in accordance with the invention may be any enzyme that can synthesize a nucleic acid molecule from a nucleic acid template, typically in the 5′ to 3′ direction, and which exhibits 5′ to 3′ exonuclease activity. Exemplary DNA polymerases (e.g., thermoresistant DNA polymerases) that may be used in the methods provided herein include, but are not limited to: Thermus aquaticus (Taq) DNA polymerase, Thermus thermophilus (Tth) DNA polymerase, Thermotoga neopolitana (Tne) DNA polymerase, and Thermotoga maritima (Tma) DNA polymerase, and mutants, and variants and derivatives thereof. DNA polymerases for use in the present teachings may be obtained commercially, for example, from Life Technologies®, Inc. New England Biolabs®, Qiagen®, Promega®, etc.

Capture Probe

The capture probe is designed to hybridize to the third segment (or to a portion thereof) of the TSIP synthetic DNA structure, i.e. comprises a sequence that is sufficiently complementary to the sequence of the third segment (or a portion thereof). In an embodiment, the capture probe comprises from about 7-8 to about 40 nucleotides, in further embodiments from about 10 to about 35 nucleotides, from about 10 to about 30 nucleotides, from about 15 to about 25 nucleotides or from about 16 to about 24 nucleotides, for example, 16, 17, 18, 19, 20, 21, 22, 23 or 24 nucleotides. In an embodiment, the capture probe further comprises a fluorophore which is suitable for FRET (i.e. which forms a FRET pair) with the fluorophore attached to the third segment (tag). In this embodiment, the detection of the presence of the target nucleic acid is performed by detecting the fluorescence signal emitted by the fluorophore present on the capture probe (which is excited by the fluorescence emitted by the fluorophore attached to third segment hybridized to the capture probe). In an embodiment, the capture probe is attached to a solid/physical support, for example a membrane, a plastic or glass slide, a chip or bead (e.g., microarray surface).

Preparation of Nucleic Acids (TSIP, Probes)

Nucleic acids can be synthesized by any method known in the art. Synthetic nucleic acids (probes and primers) are typically prepared by biological or chemical synthesis, although they can also be prepared by biological purification or degradation, e.g., endonuclease digestion. For short sequences such as the nucleic acid probes used in the present invention, chemical synthesis is frequently more economical as compared to biological synthesis. Chemical methods of polynucleotide or oligonucleotide synthesis include phosphotriester and phosphodiester methods (Narang, et al., Meth. Enzymol. (1979) 68:90) and synthesis on a support (Beaucage, et al., Tetrahedron Letters. (1981) 22:1859-1862) as well as phosphoramidate technique, Caruthers, M. H., et al., Methods in Enzymology (1988) 154:287-314 (1988), and others described in “Synthesis and Applications of DNA and RNA,” S. A. Narang, editor, Academic Press, New York, 1987, and the references contained therein.

Detection

Association of the above method(s) with a real-time fluorescence detection system may allow quantification. Digestion of the 5′ end of the TSIP synthetic DNA structure leads to F1 fluorescence increase in solution at each replicative cycle. This signal may be used for quantification. Specific identification may be obtained afterwards by hybridization of the tag segment to the capture probe. Quantification may also be possible in relation to fluorescence levels on a microarray surface. The detection of the signal may be performed using any reagents or instruments that detect a change in fluorescence from a fluorophore. Fluorescent measurements may be made using a fluorometer, plate reader with fluorescent detector or a real-time PCR thermocycler. It should be noted that new instruments are being developed at a rapid rate and any instruments could be used for the methods provided herein. In an embodiment, the hybridization is performed on a microarray slide, and the detection is performed using a suitable microarray analysis system, for example the ScanArray® 4000 or ScanArray® 5000 system from PerkinElmer®

In an embodiment, the above-mentioned method is performed in a single reaction. By single reaction it is meant that once the sample is exposed to (contacted with) the amplification and hybridization reagents, no additional reagents are needed to detect a target nucleic acid. In an exemplary embodiment of the present invention, the single reaction may occur in a single reaction vessel.

In an embodiment, the method is used to detect the presence of a plurality of target nucleic acids in a sample (multiplex). Multiplex detection is possible through the use of multiple TSIP synthetic DNA structures and spatial localization of different capture probes (i.e., located at discrete locations on a solid support), on a microarray slide for example. This technology avoids, for example, depletion of newly synthesized amplicons from the PCR/amplification reaction and produces short single stranded tags able to diffuse quickly to the microarray and allows the design of specific tags to avoid cross-hybridization and ensure comparable stringency between probes. In an embodiment, the detection is performed in a solid phase. The limit of possible targets usually associated with real time PCR is improved by transposing the detection from an aqueous phase to a solid phase where Cartesian coordinates give information on the detected target. Thus, in another aspect, the present invention provides a method for detecting a plurality of target nucleic acids in a single reaction. The method may comprise contacting a sample suspected to contain the plurality of target nucleic acids with amplification and hybridization reagents, amplifying the plurality of target nucleic acids thereby producing tag products, hybridizing the tag products to complementary capture probes and/or detecting hybridized material, whereby detection of hybridized material indicates the presence of target nucleic acids.

Sample

In accordance with the present disclosure, a sample encompasses any substance suspected of containing one or more target nucleic acids. Such substance may originate from a variety of sources. For example, a sample may be a medical/clinical sample, an environmental sample, a food sample, a laboratory sample, etc. and/or combinations thereof. In an exemplary embodiment, a sample suspected to contain one or more target nucleic acids may be obtained from any tissue/organ of any organism and/or from bodily excretions or fluids, for example, from an organism such as a human being. The sample, if need be, may be prepared using techniques known to a person skilled in the art including, without limitation, mechanical lysis, detergent extraction, sonication, electroporation, denaturants, etc., to disrupt the cells, bacteria and/or viruses and may also be purified if need be. In further embodiments, the sample may be processed to obtain an extract thereof enriched in nucleic acids, ranging from relatively crude to relatively pure nucleic acid preparations.

Kits or Systems

In another aspect, the present invention provides a target nucleic acid detection kit or system comprising a TSIP synthetic DNA structure as defined herein; and a capture probe as defined herein. In an embodiment, the kit or system further comprises: a solid support, amplification reagents, hybridization reagents, detection reagents, an amplification system and/or a detection system as defined herein. In an embodiment, the kit or system further comprises instructions for detecting the presence of a target nucleic acid in a sample, i.e. using the method defined herein. In an embodiment, the kit or system further comprises oligonucleotides (primers) hybridizing to the target nucleic acid, which may be suitable for amplification of the target nucleic acid.

### MODE(S) FOR CARRYING OUT THE INVENTION

The present invention is illustrated in further details by the following non-limiting examples.

### Example 1

### Design of an Exemplary TSIP Synthetic DNA Structure

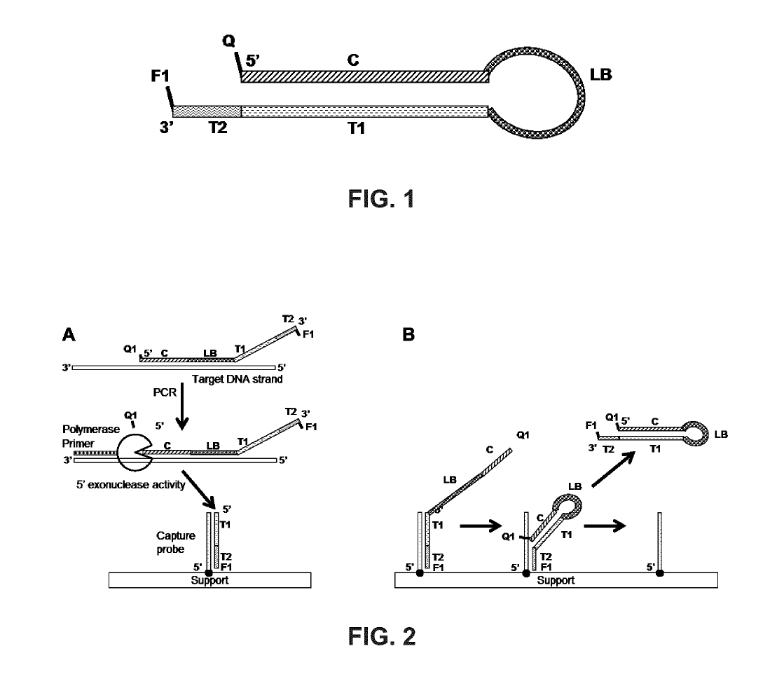

An exemplary structure of the TSIP synthetic DNA structure (nanobiomachine) is shown in FIG. 1. Complementary arm C (slash) is the reverse-complement to a target nucleic acid and T1 (dash). Loop LB (spheres) is complementary to a target nucleic acid but not to other parts of the TSIP synthetic DNA structure. T1 (dash) is complementary to C and part of a microarray capture probe. T2 (waves) is complementary to the other part of the microarray capture probe and its sequence (typically 4-9-nt) can be designed at will to ensure unique encoding for each amplicon species while ensuring optimal hybridization with immobilized capture probes. A quencher Q1 is on the 5′ end while a fluorophore F1 is on the 3′ end. The complete TSIP synthetic DNA structure can be represented by the formula Q1-C[ ]-LB[ ]-T1[ ]-T2[ ]-F1 where — represent bonds (e.g., phosphodiesters bonds and/or other covalent bonds) and [ ] are used to indicate the number of nucleotides for each segment. TABLE 1 shows exemplary TSIP synthetic DNA structure configurations and control sequences.

TABLE 1
Exemplary TSIP synthetic DNA structure configurations and control sequences

| SEQ | | CONFIGURATION | |
|---|---|---|---|
| ID NO. | NAME | FORMULA | SEQUENCE |
| 1 | Exemplary | Q-C[12]-LB[28]-T1[12]- | BHQ2*- |
| | TSIP synthetic | T2[5]-F1 | ACTCTTCAGACACGGCCTGAAAAGAGGGCCTTCTA |
| | DNA structure | | CGGAATGTCTGAAGAGTATATA-Quasar670 |
| 2 | Synthetic “always | T1[12]-T2[5]F1 | TGTCTGAAGAGTATATA-Quasar670 |
| | on” conformation | | |
| | of exemplary TSIP | | |
| | synthetic DNA | | |
| | structure | | |
| 3 | Exemplary shorter | Q-C[11]-LB[22]-T1[11]- | BHQ2- |
| | stem (C and T1) | T2[8]-F1 | ACTCTTCAGACACGGCCTGAAAAGAGGGCCTTCGT |
| | and loop TSIP | | CTGAAGAGTATATATTT-Quasar670 |
| | synthetic DNA | | |
| | structure | | |
| 4 | Synthetic “always | T1[11]-T2[8]-F1 | GTCTGAAGAGTATATATTT-Quasar670 |
| | on” conformation | | |
| | of shorter stem | | |
| | and loop of | | |
| | exemplary TSIP | | |
| | synthetic DNA | | |
| 5 | High stem-opening | Q-[3]-C[16]-LB^{a}[14]-[2]- | IAbRQ**- |
| | temperature | T1[12]-T2[8]-F1 | TATACTCTTCAGACACGGCCTGAAAAGAGGGCCCG |
| | | | TGTCTGAAGAGTATATATTT-TAMRA*** |
| 6 | Complementary | [20] | Cy5-TGTCTGAAGAGTATATATTT |
| | sequence to | | |
| | capture probe | | |
| 7 | Forward primer | [25] | GCACTTGATATTGTGGAYTMTTGAT |
| 8 | Reverse primer | [22] | GATACTCTTCCCTCATAGACTC |
| 9 | Capture probe | [20] | AAATATATACTCTTCAGACA |
| 10 | Non-specific | [20] | AGGATAGGCAGACCATACTC |
| | control probe1 | | |
| 11 | Non-specific | [20] | TACATCACGCTACGCAGTGT |
| | control probe2 | | |
| 12 | Non-specific | [20] | TTCGCTGAACAGGTAAAAGT |
| | control probe3 | | |
| 13 | Exemplary TSIP synthetic DNA structure target1 | C[17]-t(-Q1)-LB[13]- T1.1.1[3]- t(F1)-T1.1.2[4]-T2[14]- T1.2[10] | 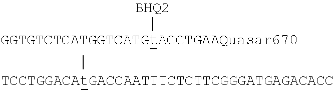 |
| 14 | Forward primer | [23] | CATGAAGCATTTGARATAGCAGA |
| | target1 | | |
| 15 | Reverse primer | [23] | CTGTGTGARTGTGATGCTTGTTT |
| | target1 | | |
| 16 | Capture probe | [17] | CATCCCGAAGAGAAATT |
| | target1 | | |
| 17 | Exemplary TSIP synthetic DNA structure target2 | C1.1[12]-t-(-Q1)- C1.2[3]-LB[13]-T1.1.1[2]- t(F1)-T1.1.2[5]-T2[14]- T1.2[8] | 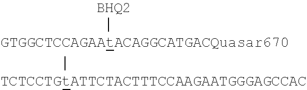 |
| 18 | Forward primer | [29] | GAATCTAGAAARTCCTACAAAAAAATGCT |
| | target2 | | |
| 19 | Reverse primer | [23] | CCTGCTGCTAATTTRGTTATTAC |
| | target2 | | |
| 20 | Capture probe | [17] | TCCCATTCTTGGAAAGT |
| | target2 | | |
| 21 | Non-specific | [17] | CTACTAAGCAGCAGGAA |
| | control probe4 | | |

-: bonds.
*BHQ2: Black Hole Quencher 2 from Biosearch
**IAbRQ: Iowa black Red Quencher from IDT
***TAMRA: tetramethylrhodamine
Small underlined “t” = thymine residue onto which the fluorophore and quencher is attached

The TSIP synthetic DNA structure has a secondary structure similar to a molecular beacon, but one side of the stem and the loop are comprised of specific DNA for recognition of the target, while the second part of the stem has an added 3′ tag that is specific to a capture probe on a microarray. The presence of the specific target nucleic acid produces a short oligonucleotide tag able to diffuse to the affixed capture probe faster than a longer amplicon. All molecular tags may be designed to hybridize at the same temperature with their complementary microarray probes. This is made possible by adjusting their Tm using the designed tag sequence T2. TABLE 2 shows melting temperature of hybridizations within the TSIP synthetic DNA structure and with target DNA or microarray capture probe.

TABLE 2
Melting temperature of hybridizations within TSIP synthetic DNA
structure and with target DNA or microarray capture probe

| | C[12]-LB[28] | C[12] | T1[12] | T1[12]-T2[5] |
|---|---|---|---|---|
| Complementary | 80.8° C.^{b} | NC | NC | NC |
| target DNA[40] | | | | |
| Complementary | NC | NC | 40° C. | 47.5° C. |
| microarray | | | | |
| capture probe[20] | | | | |
| Self- | 65.1° C.^{c} | 65.1° C.^{c} | NC | NC |
| complementary | | | | |
| T1[12] | | | | |

NC: Not complementary, no hybridization between those segments can occur.
^{b}Tm calculations using Baldino et al. (1989) equation, found in Sambrook, J., Fritsch, E. F. and Maniatis, T. Molecular Cloning: A Laboratory Manual Third edition (2001), p. 8.15 and 8.16, vol. 2, Cold Spring Harbor Laboratory Press, Cold Spring Harbor, NY
^{c}Tm calculation for molecular beacons and self-complementary nucleic acids sequences from M. Zuker, Mfold web server for nucleic acid folding and hybridization prediction. Nucleic Acids Res. 31 (13), 3406-15, (2003)

### Example 2

### Single Reaction Amplification and Hybridization Optimization

A schematic of amplification probe and target competition functions of an exemplary TSIP synthetic DNA structure (see FIG. 1) is shown in FIG. 2. In FIG. 2A an amplification target (white) is present. The TSIP synthetic DNA structure opens and the C (slashes) and LB (spheres) parts hybridize to the target. Primer (triangles) and amplification enzyme (white circle with missing pie slice) with extension and 5′ exonuclease activity recognizes the specific TSIP synthetic DNA structure-target complex. During primer extension the exonuclease moiety of the amplification enzyme encounters the TSIP synthetic DNA structure and digests the 5′ end of the C segment, releasing the quencher Q1 then completely digests the hybridized C and LB segments, freeing the tag segment comprising T1[12]-T2[5]-F1 (T1=dashes and T2=waves) (“always on” conformation). The released tag then hybridizes to a microarray capture probe (dots) and fluorescence from F1 is observed. In FIG. 2B no amplification target is present. The TSIP synthetic DNA structure is not digested. During thermal cycling, the TSIP synthetic DNA structure opens (“conditionally on” conformation) and may hybridize with the microarray capture probe (dots). There is then an equilibrium between the “conditionally on” conformation and the “off” conformation (when C[12] is hybridized to T1[12]) where quencher Q1 is in close proximity to fluorophore F1, resulting in no or low fluorescence from F1. In the “off conformation”, the tag segment T1[ ]_T2[ ]-F1 of the TSIP synthetic DNA structure is freed from the capture probe (complete quenching of F1 by proximal Q1) or is partially hybridized to the capture probe (partial quenching of F1 by a somewhat more distant Q1). Light source excites F1, F1 is either too far from the surface and efficiently quenched by proximal Q1 (freed tag segment T1[ ]-T2[ ]-F1 releasing the TSIP synthetic DNA structure from the surface) for emission of fluorescence from F1 or is close enough to Q1 for partial quenching of fluorescence. Reduced emission from F1 is observed.

For optimization of the single reaction condition, PCR mixes were made with 5 different buffers to validate compatibility with microarray hybridizations: GoTaq® (Promega®), PC2® (KlenTaq®), AptaTaq® (Roche® Applied Sciences), Takara® Taq premix (Takara Bio®) and One-step® RT-PCR reaction mix (Invitrogen®). Synthetic T1[ ]-T2[ ]-F1 oligonucleotide was used as positive control while a reaction mix containing the TSIP synthetic DNA structure without target DNA was used as a negative control. Amplifications were carried on Rotor-Gene® 3000 (Corbett Research®). An exemplary nucleic acid amplification protocol is provided below.
a. PCR amplification in one of five buffers listed above with:
   i. 3.30 mg/mL of BSA
   ii. Typically 1.50; 2.50; 3.50 or 4.50 mM of MgCl₂ (not limited to those numbers)
   iii. PCR buffer adjusted to 1× final concentration
   iv. 0.2 mM dNTPs
   v. 0.40 μM of primers SEQ ID No. 007 and 008
   vi. Typically 0.20; 0.10 or 0.05 μM of nucleic acids of SEQ ID No:1, 2, 3, 4, 5 or 6.
b. Typical cycling conditions of
   i. Temp 94.0° C., 180 s (denaturation for Hotstart)
   ii. Temp 95.0° C., 5 s (DNA denaturation)
   iii. Temp 58.0° C., 15 to 30 s (primer hybridization)
   iv. Temp 72.0° C., 20 to 30 s (elongation and TSIP synthetic DNA structure digestion)
   v. 40 to 45 cycles (ii, iii and iv)
   vi. Temp 72.0° C., 180 s (final elongation, optional step)
   vii. Temp 40.0° C. to 50.0° C., 10 mins

Hybridization was performed immediately after PCR amplification by transferring the whole reaction mixture in a HybriWell® chamber and glass slide microarray as further described below. An exemplary nucleic acid amplification protocol is provided below.
a. Microarrays are made of SEQ ID Nos: 9 to 12. Each oligonucleotide probes are typically spotted at 30 μM final concentration, making spots of ˜100 μm spaced by ˜80-100 μm on aldehyde chemistry glass slides or Zeonor® 1060R plastic slides as previously described (Zhao et al., Journal of Clinical Microbiology, 46:3752-3758 (2008) or any other microarray technology known in the art.
b. In one method, Hybriwell® chambers (Grace Biolabs®) are bonded on glass slides (Genetix®), the PCR reaction is transferred into the chamber and sealed by stickers. Slides are deposited on a heated surface at one temperature from 30 to 50° C. for about 10 mins.
c. Liquid is expulsed from slides, they are dried by a quick (10-20 seconds) centrifugation and scanned by a confocal microarray scanner
d. In another embodiment, Zeonor® slides are cut to fit inside 0.2 ml microtubes. They are inserted inside the tube before PCR amplification.
e. Afterwards, liquid is removed from tubes. Tubes are centrifuged quickly (10-20 seconds)

Zeonor® slides are read from a confocal microscope or custom setup.

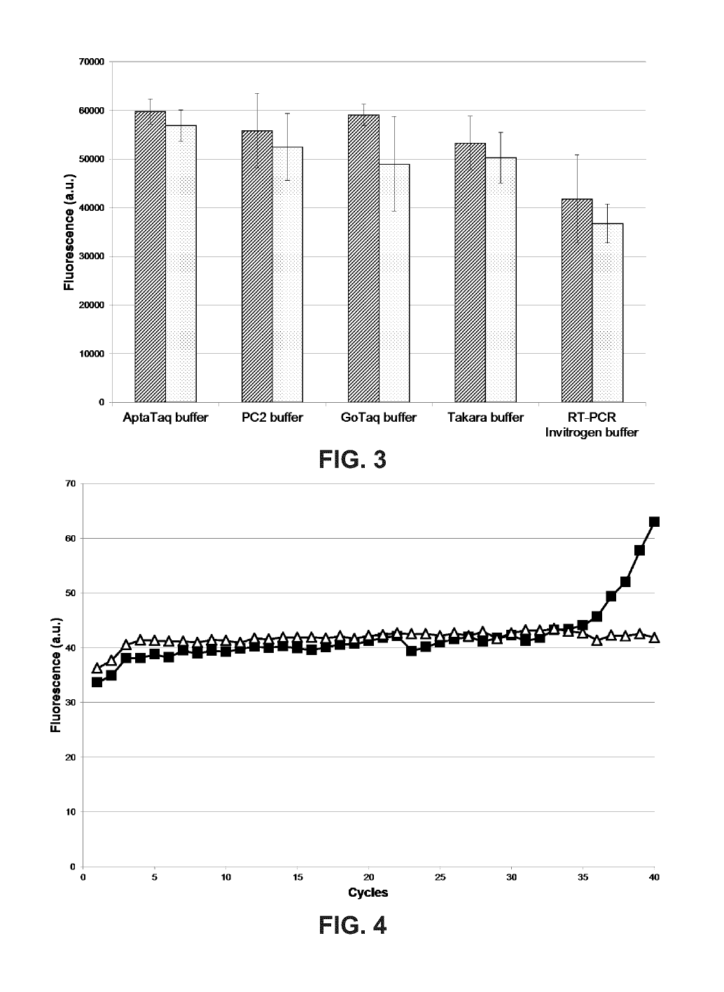

Hybridization of the synthetic T1[12]-T2[8]-F1 oligonucleotide in five amplification buffers is shown in FIG. 3. Hybridization of the synthetic T1[12]-T2[8]-F1 oligonucleotide in five amplification buffers. T1[12]-T2[8]-F1 (“always on” conformation) (slashes) and control oligonucleotide (dots) hybridization to specific capture probes. Oligonucleotides were used at 1 μM/reaction. Standard deviation on 16 or more microarray spots.

Real-time nucleic acid amplification resulting in TSIP synthetic DNA structure digestion is shown in FIG. 4. Black squares trace represents microtube containing 10⁴ copies of amplification target DNA and 0.1 μM/reaction of TSIP synthetic DNA structure. White triangles trace represents microtube without amplification target DNA and 0.1 μM/reaction of TSIP synthetic DNA structure.

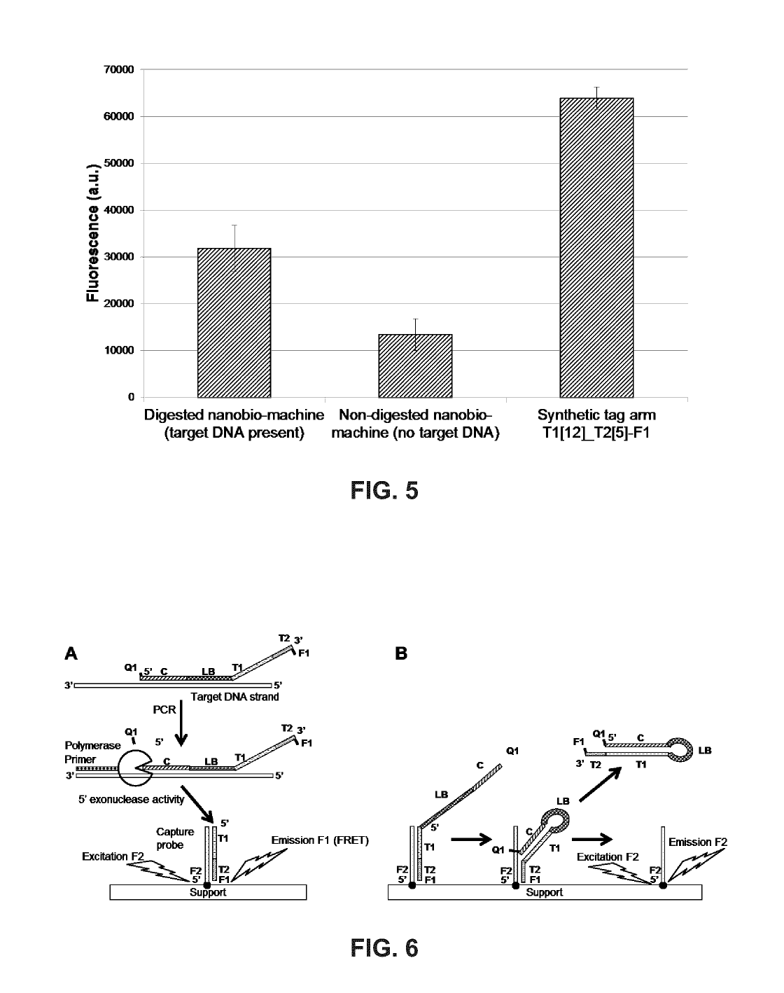

Hybridization comparison between amplification-digested TSIP synthetic DNA structure, positive and negative controls is shown in FIG. 5. All three oligonucleotide sets were submitted to thermal cycling amplification protocol in PCR buffer containing specific primers. The digested TSIP synthetic DNA structure tube contained 10⁴ copies of target DNA. The non-digested TSIP synthetic DNA structure contained no target DNA. In the final tube, the synthetic tag segment T1[ ]-T2[ ]-F1 replaced the TSIP synthetic DNA structure. Oligonucleotides were used at 0.1 μM/reaction. Standard deviation on 27 or more microarray spots.

Results show that both amplification and hybridization of oligonucleotides can be performed in 5 different amplification buffers. TSIP synthetic DNA structure is digested in presence of amplification target DNA, resulting in increased fluorescence. Hybridization of the modified form (tag segment T1[ ]-T2[ ]-F1) of the TSIP synthetic DNA structure to a specific capture probe showed a signal-to-noise ratio of 2.5-10 as compared to the non-modified form.

### Example 3

### Signal to Background Ratio Optimization

FRET Approach

To optimize signal to background ratio, the microarray-affixed capture probe is labeled with a second fluorophore. Detection of hybridized tags is achieved by FRET between the tag (fluorophore F1) and capture probe fluorophore F2, permitting specific detection of the hybridized tags. A schematic of amplification probe and target competition functions of the TSIP synthetic DNA structure using FRET is shown in FIG. 6. In FIG. 6A an amplification target (white) is present. The TSIP synthetic DNA structure opens and the C (slashes) and LB (spheres) parts hybridize to the target. Primer (triangles) and amplification enzyme (white circle with missing pie slice) with extension and 5′ exonuclease activity recognizes the specific TSIP synthetic DNA structure-target complex. During primer extension the exonuclease moiety of the amplification enzyme encounters the TSIP synthetic DNA structure and by digesting the 5′ end of the C part, it releases the quencher Q1 then completely digests the hybridized C and LB parts, freeing the tag segment comprising T1[12]-T2[5]-F1 (T1=dashes and T2=waves) (“always on” conformation). The released tag then hybridizes to a microarray capture probe (dots) labeled with fluorophore F2. Light source excites F2, which transmits its energy to F1 by a fluorescence resonance energy transfer (FRET). Emission from F1 is observed only when it is in close proximity to the excited F2. In FIG. 6B no amplification target is present. The TSIP synthetic DNA structure is not digested. During amplification, the TSIP synthetic DNA structure opens (“conditionally on” conformation) and may hybridize with the microarray capture probe labeled with fluorophore F2. There is then an equilibrium between the “conditionally on” conformation and the “off” conformation (when C[12] is hybridized to T1[12]) where quencher Q1 is in close proximity to fluorophore F1. In the “off conformation”, the tag segment T1[ ]-T2[ ]-F1 from the TSIP synthetic DNA structure is freed from the capture probe (complete quenching of F1 by proximal Q1) or is partially hybridized to the capture probe (partial quenching of F1 by a somewhat more distant Q1). Light source excites F2, F1 is either too far (TSIP synthetic DNA structure tag segment T1[ ]-T2[ ]-F1 is freed from capture probe, releasing the TSIP synthetic DNA structure from the microarray surface) for FRET or is close enough to Q1 for partial quenching of fluorescence (from F1 and F2). Emission from F2 is observed or greatly reduced emission from F1 is observed.

Modification of Undigested TSIP Synthetic DNA Structure Approach

In an exemplary embodiment, psoralen and/or UV exposure may be used to inactivate undigested TSIP synthetic DNA structure resulting in crosslinking C[ ] to T1[ ] so that it remains in the “always off” conformation. It should be used at temperatures higher than the Tm of T1[ ]-T2[ ]+ capture probe and lower than the Tm of the stem segment (C[ ] hybridized to T1[ ].

### Example 4

### Negative Sample Determination

In an embodiment, real-time fluorescence may be used to monitor probe conversion into single-stranded tags allowing faster determination of negative samples. This would remove the need for reading the microarray if no fluorescence increase has been measured during the amplification.

### Example 5

### SNP Detection

SNP detection and discrimination is done in solution by precise recognition of the target by the TSIP synthetic DNA structure. Discrimination by the TSIP synthetic DNA structure can be enhanced by using nucleotide modifications known to increase this discrimination such as locked nucleic acid (LNA) or equivalent. Two different SNPs will trigger the release of two completely different tags which will hybridize at two different positions onto the microarray.

### Example 6

### Other TSIP Configurations

Another exemplary TSIP synthetic DNA structure may take the following configuration represented by the formula:

Q1-C[ ]-LB[ ]-T2[ ]-T1[ ]-F1 where

where —, Q1, C, [ ], LB, T2, T1 and F1 are as defined in example 2.

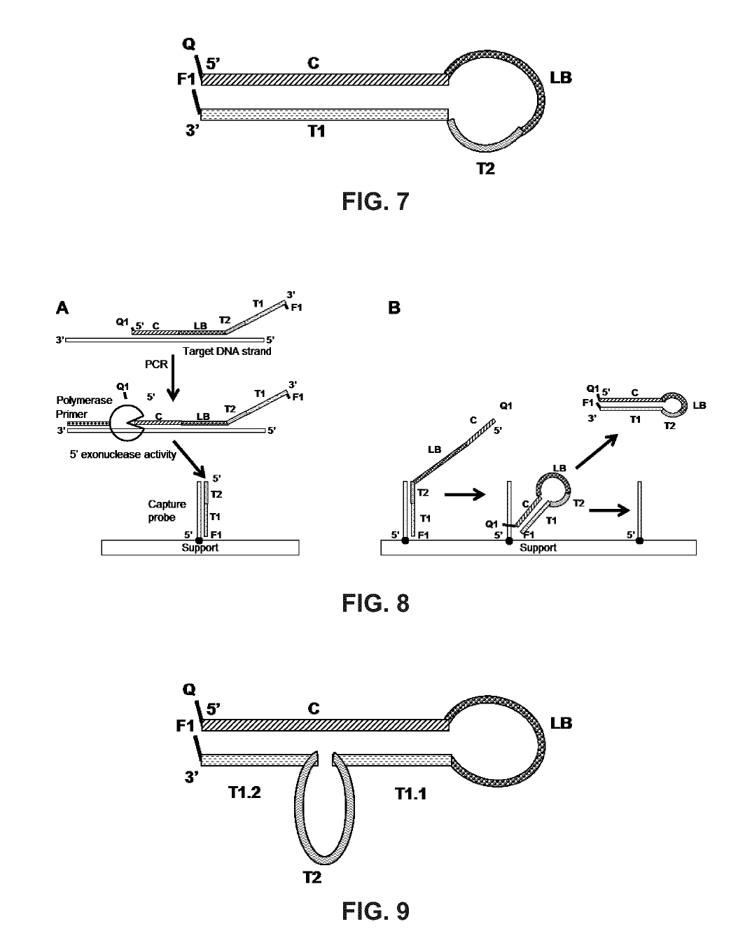

This conformation is shown in FIG. 7 where complementary arm C (slash) is the reverse-complement to a target nucleic acid and T1 (dash). Loop LB (spheres) is complementary to a target nucleic acid but not to other parts of the TSIP synthetic DNA structure. Loop T2 (waves) (1 to 9 nt) is complementary to a part of the microarray capture probe and may be designed at will to ensure unique encoding for each amplicon species while ensuring optimal hybridization with immobilized capture probes. T1 (dash) is complementary to C and part of the capture probe. A quencher Q1 is on the 5′ end while a fluorophore F1 is on the 3′ end.

A schematic of amplification probe and target competition functions of this configuration is shown in FIG. 8. In FIG. 8A, an amplification target (white) is present, the TSIP synthetic DNA structure opens and the C (slashes) and LB (spheres) parts hybridize to the target. Primer (triangles) and amplification enzyme (white circle with missing pie slice) with extension and/or 5′ exonuclease activity recognizes the specific TSIP synthetic DNA molecule-target complex. During primer extension the exonuclease moiety of the amplification enzyme encounters the TSIP synthetic DNA structure and by digesting the 5′ end of the C part, it releases the quencher Q1 then completely digests the hybridized C and LB parts, freeing the tag segment comprising T2[1-9]-T1[12]-F1 (T1=dashes and T2=waves) (“always on” conformation). The released tag then hybridizes to a microarray capture probe (dots) and fluorescence from F1 is observed. In FIG. 8B no amplification target is present. TSIP synthetic DNA structure is not digested. During thermal cycling, the TSIP synthetic DNA structure opens (“conditionally on” conformation) and may hybridize with the microarray capture probe (dots). There is then an equilibrium between the “conditionally on” conformation and the “off” conformation (when C[12] is hybridized to T1[12]) where quencher Q1 is in close proximity to fluorophore F1, resulting in no or low fluorescence from F1. In the “off conformation”, the TSIP synthetic DNA structure is freed from the capture probe leading to quenching of F1 by proximal Q1 or is partially hybridized to the capture probe leading to less efficient quenching of F1 by a somewhat more distant Q1. Light source excites F1, F1 is either efficiently quenched by proximal Q1 (freed TSIP synthetic DNA structure) thus preventing emission of fluorescence from F1 or is not close enough to Q1 for efficient quenching of fluorescence. Reduced emission from F1 is observed. FRET is also possible in this conformation.

Another exemplary TSIP synthetic DNA structure may take the configuration represented by the formula:

Q1-C[ ]-LB[ ]-T1.1[ ]-T2[ ]-T1.2[ ]-F1 where

T1.1 segment is complementary to part of the C segment,

T1.2 segment is complementary to part of the C segment,

—, Q1, C, [ ], LB, T2, T1 and F1 are as defined in example 2.

This conformation is shown in FIG. 9 where the complementary arm C (slash) is the reverse-complement to a target nucleic acid and T1.1 and T1.2 (dash). Loop LB (spheres) is complementary to a target nucleic acid but not to other parts of the TSIP synthetic DNA structure. T1 (dash) is complementary to C but separated in two segments (T1.1 and T1.2) by T2. T2 (waves) is only complementary to the microarray capture probe and its sequence (typically 15-20 nt) may be designed at will to ensure unique encoding for each amplicon species while ensuring optimal hybridization with immobilized capture probes. A quencher Q1 is on the 5′ end while a fluorophore F1 is on the 3′ end. In this configuration, only T2 is complementary to the capture probe. It may be designed at will to ensure specificity and optimal hybridization conditions.

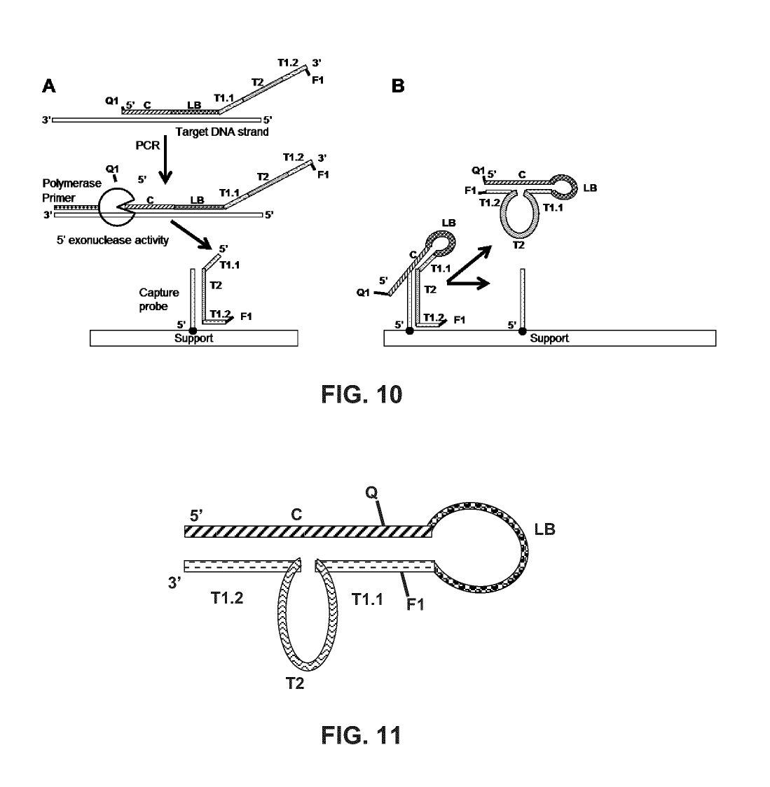

A schematic of amplification probe and target competition functions of this conformation is shown in FIG. 10. In FIG. 10A an amplification target (white) is present, the TSIP synthetic DNA structure opens and the C (slashes) and LB (spheres) parts hybridize to the target. Primer (triangles) and amplification enzyme (white circle with missing pie slice) with extension and/or 5′ exonuclease activity recognizes the specific TSIP synthetic DNA structure-target complex. During primer extension the exonuclease moiety of the amplification enzyme encounters the TSIP synthetic DNA structure and by digesting the 5′ end of the C part, it releases the quencher Q1 then completely digests the hybridized C and LB parts, freeing the loop T2 sandwiched between T1.1 and T1.2. (T1=dashes and T2=waves) (“always on” conformation). The released tag then hybridizes to a microarray capture probe (dots) and fluorescence from F1 is observed. In FIG. 10B no amplification target is present; the TSIP synthetic DNA molecule is not digested. During thermal cycling, the TSIP synthetic DNA structure opens (“conditionally on” conformation) and may hybridize with the microarray capture probe (dots). C stays hybridized to T1.1. There is then an equilibrium between the “conditionally on” conformation and the “off” conformation (when C[12] is hybridized to T1.1+T1.2[12 to 20]) where quencher Q1 is in close proximity to fluorophore F1, resulting in no or low fluorescence from F1. In the “off conformation”, the TSIP synthetic DNA structure is freed from the capture probe (complete quenching of F1 by proximal Q1) or is partially hybridized to the capture probe (partial quenching of F1 by a somewhat more distant Q1). Light source excites F1, F1 is either too far from the surface and efficiently quenched by proximal Q1 (freed TSIP synthetic DNA structure) for emission of fluorescence from F1 or is close enough to Q1 for partial quenching of fluorescence. No or reduced emission from F1 is observed. FRET is also possible in this configuration.

### Example 7

### Single Vessel for Both Amplification and Hybridization

A schematic of amplification probe and target competition functions of an exemplary TSIP synthetic DNA structure (see FIG. 1) is shown in FIG. 2. In FIG. 2A an amplification target (white) is present. The TSIP synthetic DNA structure opens and the C (slashes) and LB (spheres) parts hybridize to the target. Primer (triangles) and amplification enzyme (white circle with missing pie slice) with extension and 5′ exonuclease activity recognizes the specific TSIP synthetic DNA structure-target complex. During primer extension the exonuclease moiety of the amplification enzyme encounters the TSIP synthetic DNA structure and digests the 5′ end of the C segment, releasing the quencher Q1 then completely digests the hybridized C and LB segments, freeing the tag segment comprising T1[12]-T2[5]-F1 (T1=dashes and T2=waves) (“always on” conformation). The released tag then hybridizes to a microarray capture probe (dots) and fluorescence from F1 is observed. In FIG. 2B no amplification target is present. The TSIP synthetic DNA structure is not digested. During thermal cycling, the TSIP synthetic DNA structure opens (“conditionally on” conformation) and may hybridize with the microarray capture probe (dots). There is then an equilibrium between the “conditionally on” conformation and the “off” conformation (when C[12] is hybridized to T1[12]) where quencher Q1 is in close proximity to fluorophore F1, resulting in no or low fluorescence from F1. In the “off conformation”, the tag segment T1[ ]-T2[ ]-F1 of the TSIP synthetic DNA structure is freed from the capture probe (complete quenching of F1 by proximal Q1) or is partially hybridized to the capture probe (partial quenching of F1 by a somewhat more distant Q1). Light source excites F1, F1 is either too far from the surface and efficiently quenched by proximal Q1 (freed tag segment T1[ ]-T2[ ]-F1 releasing the TSIP synthetic DNA structure from the surface) for emission of fluorescence from F1 or is close enough to Q1 for partial quenching of fluorescence. Reduced emission from F1 is observed.

PCR amplification was performed in the presence of a Zeonor® 1060R microarray inside the reaction vessel. GoTaq® (Promega®) enzyme and buffer was used for the amplification. A reaction mix containing the TSIP synthetic DNA structure without target DNA was used as a negative control. Amplifications were carried as described in example 2

Hybridization was performed as described in Example 2, more specifically steps d and e of the hybridization procedure described in Example 2 were used.

Hybridization of the TSIP of SEQ ID NO: 1 in a single vessel for both amplification and hybridization shown in FIG. 13. White bar: negative samples. Grey bar: samples containing the target. Fluorescence signal of the digested TSIP is compared to the non-digested negative control and gives a ratio of 2.55.

### Example 8

### Single Vessel Multiplexed Amplification and Hybridization

Another exemplary TSIP synthetic DNA structure may take the configuration represented by the formula:

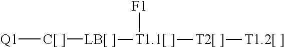

where

—, Q1, C, [ ], LB, T1.1, F1, T2 and T1.2 are as defined in Examples 2 and 6.

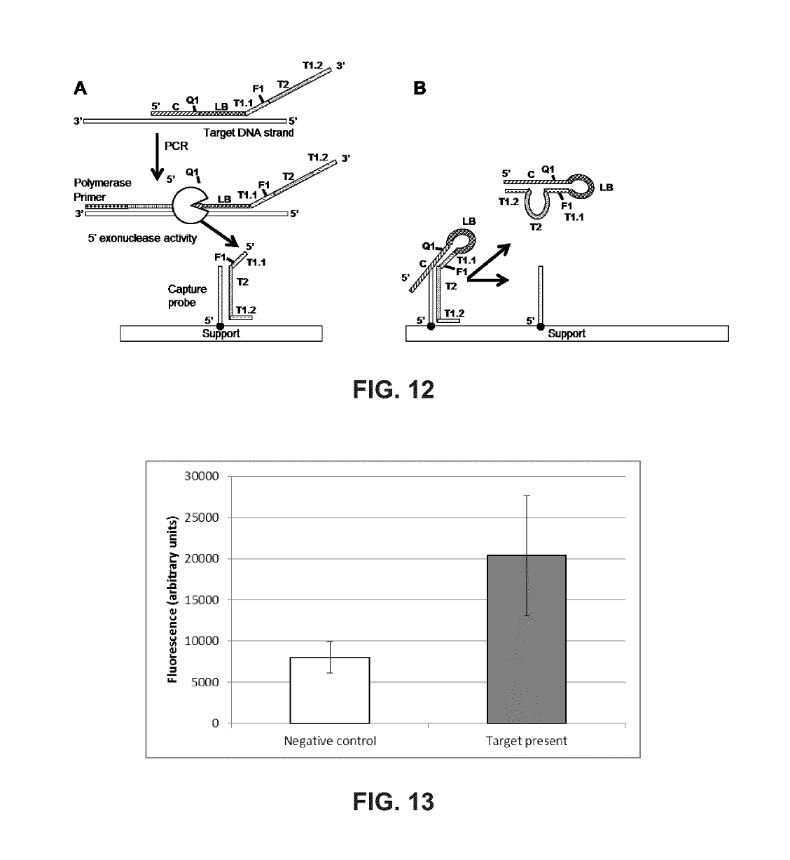

A schematic of amplification probe and target competition functions of an exemplary TSIP synthetic DNA structure (see FIG. 11) is shown in FIG. 12A. In FIG. 12A an amplification target (white) is present. The TSIP synthetic DNA structure opens and the C (slashes) and LB (spheres) parts hybridize to the target. Primer (triangles) and amplification enzyme (white circle with missing pie slice) with extension and 5′ exonuclease activity recognizes the specific TSIP synthetic DNA structure-target complex. During primer extension the exonuclease moiety of the amplification enzyme encounters the TSIP synthetic DNA structure and digests the 5′ end of the C segment, releasing the quencher Q1 then completely digests the hybridized C and LB segments, freeing the tag segment comprising F1-T1.1[ ]-T2[ ]-T1.2[ ] (T1=dashes and T2=waves) (“always on” conformation). The released tag then hybridizes to a microarray capture probe (dots) and fluorescence from F1 is observed. In FIG. 12B no amplification target is present for the second specific TSIP. The TSIP synthetic DNA structure is not digested. During thermal cycling, the TSIP synthetic DNA structure opens (“conditionally on” conformation) and may hybridize with the microarray capture probe (dots). There is then an equilibrium between the “conditionally on” conformation and the “off” conformation (when C[ ] is hybridized to T1.1[ ]+T1.2 [ ]) where quencher Q1 is in close proximity to fluorophore F1, resulting in no or low fluorescence from F1. In the “off conformation”, the tag segment F1-T1.1[ ]-T2[ ]-T1.2[ ] of the TSIP synthetic DNA structure is freed from the capture probe (complete quenching of F1 by proximal Q1) or is partially hybridized to the capture probe (partial quenching of F1 by a somewhat more distant Q1). Light source excites F1, F1 is either too far from the surface and efficiently quenched by proximal Q1 (freed tag segment F1-T1.1[ ]-T2[ ]-T1.2[ ] releasing the TSIP synthetic DNA structure from the surface) for emission of fluorescence from F1 or is close enough to Q1 for partial quenching of fluorescence. No emission or reduced emission from F1 is observed.

PCR amplification was performed using GoTaq® (Promega®) enzyme and buffer for the amplification. A reaction mix containing 2 different TSIP synthetic DNA structures without target DNA was used as a negative control. Amplifications were carried on Rotor-Gene® 3000 (Corbett Research®). An exemplary nucleic acid amplification protocol is provided below.
c. PCR amplification the buffer listed above with:
   i. 3.30 mg/mL of BSA
   ii. Typically 1.50; 2.50; 3.50 or 4.50 mM of MgCl₂ (not limited to those numbers)
   iii. PCR buffer adjusted to 1× final concentration
   iv. 0.2 mM dNTPs
   v. 0.40 μM of primers SEQ ID No. 14-15 and 18-19
   vi. Typically 0.20; 0.10 or 0.05 μM of nucleic acids of SEQ ID NO: 13 and 17.
   vii. Addition of either no target, target1 or target2.
d. Typical cycling conditions of
   i. Temp 94.0° C., 180 s (denaturation for Hotstart)
   ii. Temp 95.0° C., 5 s (DNA denaturation)
   iii. Temp 58.0° C., 15 to 30 s (primer hybridization)
   iv. Temp 72.0° C., 20 to 30 s (elongation and TSIP synthetic DNA structure digestion)
   v. 40 to 45 cycles (ii, iii and iv)
   vi. Temp 72.0° C., 180 s (final elongation, optional step)
   vii. Insertion of microarrays into the reaction vessel
   viii. Temp 40.0° C. to 50.0° C., 10 mins

Hybridization was performed at the end of the PCR cycle (previously described in vii.).
f. Microarrays are made of SEQ ID NOs: 16, 20 and 21. Each oligonucleotide probes are typically spotted at 30 μM final concentration, making spots of ˜100 μm spaced by ˜80-100 μm on Zeonor® 1060R plastic slides (Zhao Z et al., 2008 supra).
g. Zeonor® 1060R slides are cut to fit inside 0.2 ml microtubes. They are inserted inside the tube immediately after PCR amplification.
h. Afterwards, liquid is removed from tubes. Tubes are centrifuged quickly (10-20 seconds)

Zeonor® slides are read from a confocal microscope or custom setup.

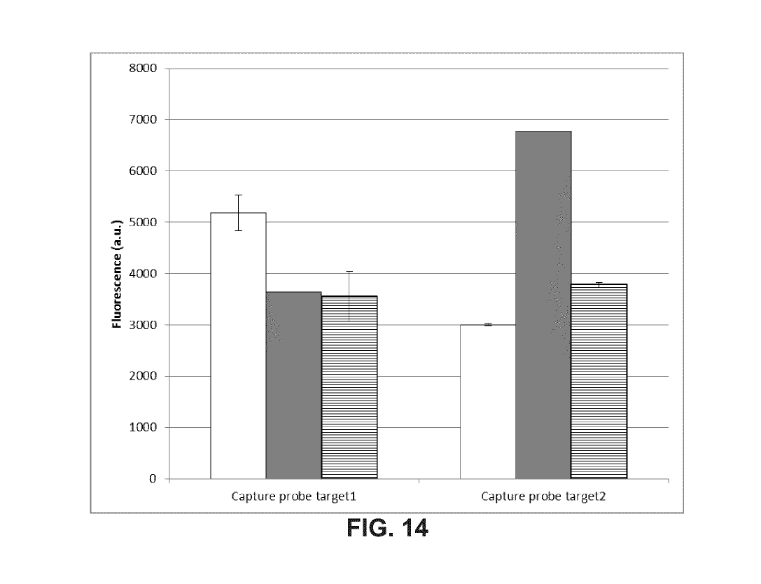

Hybridization of the TSIP of SEQ ID NOs: 13 (target1) and 17 (target2) in a single vessel for both amplification and hybridization is shown in FIG. 14. White bar: amplification/hybridization was carried on a positive sample for target1. Grey bar: amplification/hybridization was carried on a positive sample for target2. Horizontal lines bar: amplification/hybridization was carried in absence of both target1 and target2. Results of fluorescence signal on the specific capture probe for one target is compared to the fluorescence signal on the other capture probe. For target1, a ratio of 1.72 was observed while a ratio of 1.86 was observed for target2.

Although the present invention has been described hereinabove by way of specific embodiments thereof, it can be modified, without departing from the spirit and nature of the subject invention as defined in the appended claims.

1. A method for detecting the presence of a target nucleic acid in a sample, the method comprising:
(a) contacting a sample with amplification and hybridization reagents, wherein said amplification and hybridization reagents comprise a target specific indexing probe (TSIP) synthetic DNA structure comprising a first segment, a second segment and a third segment, wherein:
(i) the first segment comprises a sequence complementary to a first portion of the target nucleic acid and complementary to the third segment;
(ii) the second segment is attached to the 3′ end of the first segment and comprises a sequence that is complementary to a second portion of the target nucleic acid that is contiguous to the first portion, but not complementary to the first or third segment;
(iii) the third segment is attached to the 3′ end of the second segment and comprises
a first part comprising a sequence that is complementary to the first segment and to a capture probe present in said reaction;
a first part comprising a sequence that is complementary to the first segment, and a second part comprising a sequence that is complementary to the capture probe;
or
a first part comprising a sequence that is complementary to the first segment and to a first portion of the capture probe, and a second part comprising a sequence that is complementary to a second portion of the capture probe,
wherein the third segment comprises a detectable label attached thereto, and said first and/or second segment comprises a quenching moiety for the detectable label,
wherein the detectable label and the quenching moiety are located within sufficiently close proximity of each other in the TSIP synthetic DNA structure such that the signal emitted by the detectable label is reduced as compared to a corresponding signal emitted by a detectable label in a corresponding TSIP synthetic DNA structure lacking said quencher moiety;
(b) performing an amplification and hybridization reaction, wherein said third segment is released from the first and second segment if the target nucleic acid is present; and
(c) detecting the hybridization of the third segment to the capture probe by virtue of an increase in the signal emitted from the detectable moiety relative to the signal emitted from a corresponding TSIP that has not been contacted with the sample, wherein the detection of an hybridization is indicative that the target nucleic acid is present in the sample.

2. The method of claim 1, wherein said first segment comprises from about 8 to about 20 nucleotides.

3. (canceled)

4. The method of claim 1, wherein said second segment comprises from about 8 to about 40 nucleotides.

5. (canceled)

6. The method of claim 1, wherein said first and second segments comprise from about 16 to about 60 nucleotides in total.

7-8. (canceled)

9. The method of claim 1, wherein said third segment comprises from about 8 to about 30 nucleotides.

10. (canceled)

11. The method of claim 1, wherein said capture probe comprises from about 10 to about 30 nucleotides.

12. (canceled)

13. The method of claim 1, wherein said quenching moiety is attached to the first segment.

14-15. (canceled)

16. The method of claim 1, wherein said detectable label is located at 20 nucleotides or less from the quencher.

17. (canceled)

18. The method of claim 1, wherein the detectable label and the quenching moiety are at a distance of about 10 nm or less.

19. The method of claim 1, wherein said third segment comprises a first part comprising a sequence that is complementary to the first segment and to a first portion of the capture probe, and a second part comprising a sequence that is complementary to a second portion of the capture probe.

20. The method of claim 19, wherein said second part is attached to the 3′ end of said first part, and said TSIP synthetic DNA structure is of formula I, II, III or IV: 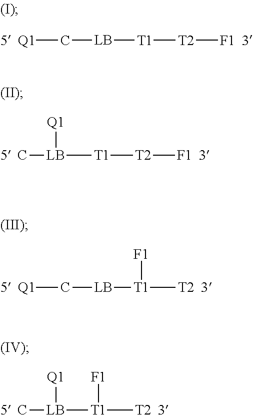
where
— represent a bond,
Q1 represents quenching moiety,
C represents said first segment,
LB represents said second segment,
T1 represents the first part of the third segment,
T2 represents the second part of the third segment, and
F1 represents the detectable label.

21. The method of claim 19, wherein said second part is attached to the 5′ end of said first part, and said TSIP synthetic DNA structure is of formula V, VI, VII or VIII: 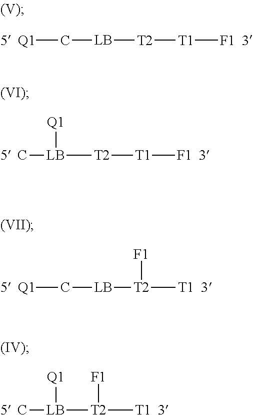
where —, Q1, C, LB, T1, T2 and F2 are as defined in claim 19.

22. The method of claim 19, wherein said second part is inserted within said first part, and said TSIP synthetic DNA structure is of formula IX, X, XI, XII, XIII or XIV 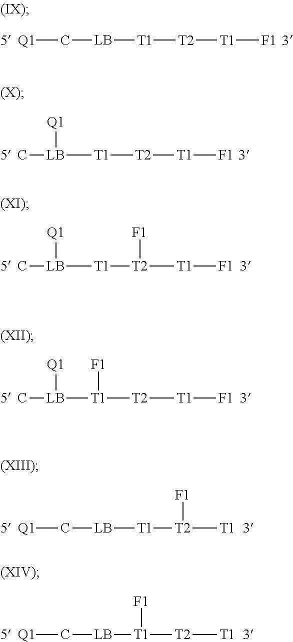
where —, Q1, C, LB, T1, T2 and F2 are as defined in claim 19.

23. The method of claim 1, wherein said detectable label is a fluorophore.

24-27. (canceled)

28. The method of claim 23, wherein said capture probe comprises a fluorophore suitable for fluorescence resonance energy transfer (FRET) with the fluorophore attached to said third segment.

29. The method of claim 1, wherein said amplification and hybridization reagents further comprise an amplification enzyme with 5′ exonuclease activity.

30. The method of claim 1, wherein said third segment further comprises a sequence recognized by a restriction endonuclease, and wherein said amplification and hybridization reagents further comprise said restriction endonuclease.

31. The method of claim 1, wherein said capture probe is attached to a solid support.

32-33. (canceled)

34. A target nucleic acid detection kit or system comprising: (a) the target specific indexing probe (TSIP) synthetic DNA structure defined in claim 1.

35. The target nucleic acid detection kit or system of claim 34, further comprising a capture probe comprising a sequence complementary to at least a portion of the third segment of the TSIP synthetic DNA structure.

36-38. (canceled)


SEQUENCE LISTING (21 sequences)

SEQ ID NO: 1 - DNA, 57 bp, Artificial Sequence
  Synthetic oligonucleotide
actcttcagacacggcctgaaaagagggccttctacggaatgtctgaagagtatata

SEQ ID NO: 2 - DNA, 17 bp, Artificial Sequence
  Synthetic oligonucleotide
tgtctgaagagtatata

SEQ ID NO: 3 - DNA, 52 bp, Artificial Sequence
  Synthetic oligonucleotide
actcttcagacacggcctgaaaagagggccttcgtctgaagagtatatattt

SEQ ID NO: 4 - DNA, 19 bp, Artificial Sequence
  Synthetic oligonucleotide
gtctgaagagtatatattt

SEQ ID NO: 5 - DNA, 54 bp, Artificial Sequence
  Synthetic oligonucleotide
tatactcttcagacacggcctgaaaagagggcccgtgtctgaagagtatatatt

SEQ ID NO: 6 - DNA, 20 bp, Artificial Sequence
  Synthetic oligonucleotide
tgtctgaagagtatatattt

SEQ ID NO: 7 - DNA, 25 bp, Artificial Sequence
  Synthetic oligonucleotide
gcacttgatattgtggaytmttgat

SEQ ID NO: 8 - DNA, 22 bp, Artificial Sequence
  Synthetic oligonucleotide
gatactcttccctcatagactc

SEQ ID NO: 9 - DNA, 20 bp, Artificial Sequence
  Synthetic oligonucleotide
aaatatatactcttcagaca

SEQ ID NO: 10 - DNA, 20 bp, Artificial Sequence
  Synthetic oligonucleotide
aggataggcagaccatactc

SEQ ID NO: 11 - DNA, 20 bp, Artificial Sequence
  Synthetic oligonucleotide
tacatcacgctacgcagtgt

SEQ ID NO: 12 - DNA, 20 bp, Artificial Sequence
  Synthetic oligonucleotide
ttcgctgaacaggtaaaagt

SEQ ID NO: 13 - DNA, 63 bp, Artificial Sequence
  Synthetic oligonucleotide
ggtgtctcatggtcatgtacctgaatcctggacatgaccaatttctcttcgggatgagacacc

SEQ ID NO: 14 - DNA, 23 bp, Artificial Sequence
  Synthetic oligonucleotide
catgaagcatttgaratagcaga

SEQ ID NO: 15 - DNA, 23 bp, Artificial Sequence
  Synthetic oligonucleotide
ctgtgtgartgtgatgcttgttt

SEQ ID NO: 16 - DNA, 17 bp, Artificial Sequence
  Synthetic oligonucleotide
catcccgaagagaaatt

SEQ ID NO: 17 - DNA, 59 bp, Artificial Sequence
  Synthetic oligonucleotide
gtggctccagaatacaggcatgactctcctgtattctactttccaagaatgggagccac

SEQ ID NO: 18 - DNA, 29 bp, Artificial Sequence
  Synthetic oligonucleotide
gaatctagaaartcctacaaaaaaatgct

SEQ ID NO: 19 - DNA, 23 bp, Artificial Sequence
  Synthetic oligonucleotide
cctgctgctaatttrgttattac

SEQ ID NO: 20 - DNA, 17 bp, Artificial Sequence
  Synthetic oligonucleotide
tcccattcttggaaagt

SEQ ID NO: 21 - DNA, 17 bp, Artificial Sequence
  Synthetic oligonucleotide
ctactaagcagcaggaa